(19) United States
(12) Patent Application Publication
(10) Pub. No.: US 2011/0054034 A1
(43) Pub. Date: Mar. 3, 2011

(54) METHODS OF USING CARBOXYLIC AMIDES AS ANTIMICROBIAL AGENTS

(76) Inventors: William Schroeder, JR., Muskegon, MI (US); Robert P. Smart, Zeeland, MI (US); Roderick M. Morgan, Allendale, MI (US); Arti J. Walker, Grand Rapids, MI (US)

(21) Appl. No.: 12/866,408

(22) PCT Filed: Feb. 6, 2009
(86) PCT No.: PCT/US2009/033379

§ 371 Date: Nov. 12, 2010

Related U.S. Application Data
(60) Provisional application No. 61/027,129, filed on Feb. 8, 2008.

(51) Int. Cl. A61K 31/165; A61P 31/00; A61P 31/04

(52) U.S. Cl. 514/621

(57) ABSTRACT

A method of treating a disease or condition in a warm blooded mammal which disease or condition is suspected to be associated with a microbial infection, comprising: administering an antimicrobial effective amount of a carboxylic acid amide, its isomers or salts thereof, to a subject who is suspected to be infected with a microbe. In one embodiment, the carboxylic acid amide is trans-3-(naphth-2-yl)-but-2-enoic acid-N-(2-carboxyphenyl)amide.

### FIELD OF THE INVENTION

The present invention is a method of using known compounds to treat systemic and topical microbial infections in useful warm blooded mammals.

### BACKGROUND OF THE INVENTION

Microbial infections of many types (e.g., bacterial, fungal, protozoan, and viral) can be the cause of a disease in mammals. Various agents to combat such infections have been developed, well known and are in use. However, due to the prevalence of such infections, there is a need for new treatments for targeting microbes. Such new agents might be less costly, more effective in treating the target infection, or capable of use in combination with another drug; or such new agents might induce less side effects than currently available compounds.

Further, many currently available anti-microbial agents or compounds have become ineffective because of resistance to the compound developed by the targeted microorganism. Organisms can become resistant through one of three general mechanisms: 1) efflux of the compound from the cell (the cell pumps the drug out of the cell before it does any damage, 2) the cell modifies the cellular target of the compound, or 3) the cell modifies the compound to a non-toxic form of the compound. Known antibiotic-resistant strains of bacteria include: Methicillin-Resistant Staphylococcus aureus (MRSA), Vancoymicin-Resistant Enterococci (VRE), and Extreme Drug Resistant Tuberculosis (XDR). Thus, a new anti-microbial agent that is effective against these and other resistant strains of bacteria also is needed.

### SUMMARY OF THE INVENTION

The present invention includes the use of various carboxylic acid amides, their isomers and salts, in treating a disease or condition in a useful warm blooded mammal who has a microbial infection that is susceptible to treatment and who is in need of treatment. The treatment is with an antimicrobial effective among of a carboxylic acid amide of this invention. In one embodiment of the present invention, carboxylic acid amides are used to inhibit the growth of or to kill microbes that are otherwise resistant to antimicrobial agents.

The present invention includes a method of treating a disease or condition in a useful warm blooded mammal who has a susceptible microbial infection and is in need of treatment with an antimicrobial effective amount of a carboxylic acid amide including an effective amount of trans-3-(naphth-2-yl)-but-2-enoic acid-N-(2-carboxy-phenyl)-amide, its isomers or salts, to a subject who is suspected to be infected with a microbe. In one embodiment of the invention, the microbe is selected from the group consisting of Staphylococcus aureus, Bacillus subtilis, Staphylococcus epidermidis, Streptococcus pneumoniae, Micrococcus leuteus, and Mycobacterium smegmatis. In another embodiment, the microbe is Staphylococcus aureus or, more specifically methicillin resistant S. aureus, MRSA. Also, with this method, the subject may be human.

These and other features, advantages and objects of the present invention will be further understood and appreciated by those skilled in the art by reference to the remainder of the specification and claims.

### DETAILED DESCRIPTION OF THE PREFERRED EMBODIMENTS

The preferred embodiments of the present invention may be understood more readily by reference to the following detailed description of the specific embodiments and the Examples included hereafter.

All references, patents, patent publications, articles, and databases, referred to in this application are incorporated-by-reference in their entirety, as if each were specifically and individually incorporated herein by reference.

Unless defined otherwise, all technical and scientific terms used herein have the meaning commonly understood by one of ordinary skill in the art to which this invention belongs.

As used in this specification and the appended claims, the singular forms “a”, “an”, and “the” include plural references unless the context clearly indicates otherwise.

It will be understood by those skilled in the art that, in general, terms used herein, are generally intended as “open” terms (e.g., the term “including” should be interpreted as “including but not limited to,” the term “having” should be interpreted as “having at least,” the term “includes” should be interpreted as “includes but is not limited to,” etc.).

As used herein, the term “an effective amount” means an amount of a carboxylic acid amide that produces a statistically significant result. For example, with respect to the present invention, an effective amount of carboxylic acid amide, analog, or derivative means an amount that provides a statistically significant reduction in (a) growth of a microbe, (b) proliferation of a microbe, (c) the number of living microbes and/or (d) treats the condition or disease of the useful warm blooded mammal. Treat or treatment as used herein includes both prevention of a disease and treatment of an existing disease. An effective amount can be determined using known techniques, depending upon variables such as the particular microbe, the patient, the severity of the patients condition, the method of administration, the formulation, and other factors as is known to those skilled in the art. An effective amount is demonstrated by a statistically significant difference in growth, proliferation, or cell count for a microbe as measured between a treatment group and a control group as well as by successful treatment of a warm blooded mammal who has a susceptible microbial infection and is in need of treatment. “Subject” or “patient” refers to a useful warm blooded mammal which is selected from the group consisting of humans, horses, sheep, cattle, pigs, cats and dogs. It is preferred that the useful warm blooded mammal be a human.

As used herein, the terms “treatment” or “treating” or “treat” includes treating exixting microbial infections both topical and systemic as well as preventing such infections.

In the following description, reference will be made to various methodologies known to those of skill in the art of cell biology and molecular biology. Publications and other materials setting forth such known methodologies to which reference is made are incorporated herein by reference in their entireties as though set forth in full.

U.S. Pat. Nos. 6,362,210; 6,492,547; 6,727,250, and 6,660,764 (which patents are incorporated herein in their entirety as though set forth in full) disclose various carboxylic acid amides which are inhibitors of an enzyme named telomerase. These carboxylic acid amides are currently undergoing clinical testing as anti-cancer drugs. The carboxylic acid amides were also disclosed as being used to treat other diseases which have an increased rate of cell division or increased telomerase activity, such as epidermal hyperproliferation (psoriasis), inflammatory processes (rheumatoid arthristis), diseases of the immune system. Further, they were disclosed as being useful for treating parasitic diseases in man such as worm or fungal diseases as well as diseases caused by protozoan pathogens.

As described below, various carboxylic acid amides also are used as antimicrobial agents in the method of the present invention. Such carboxylic acid amides include those of general formula I:

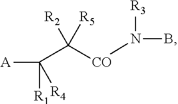

the isomers thereof, the trans-isomers thereof, and the salts thereof.

In the above general formula I, R₁ is a hydrogen atom, a C₁₋₃-alkyl or trifluoromethyl group, R₂ is a hydrogen, fluorine, chlorine or bromine atom, a C₁₋₃-alkyl, C₃₋₇-cycloalkyl or C₁₋₃-alkoxy group or also, if R₄ and R₅ each are a hydrogen atom, R₁ and R₂ together are an n-C₁₋₃-alkylene group which may be substituted by a C₁₋₃-alkyl group, R₃ is a hydrogen atom or a C₅-alkyl group, R₄ and R₅ each are a hydrogen atom or together indicate another carbon-carbon bond, A is a phenyl, naphthyl or tetrahydronaphthyl group substituted by a fluorine, chlorine, bromine or iodine atom, by a C₁₋₆-alkyl, C₃₋₇-cycloalkyl, phenyl, C₁₋₃-alkoxy, cyano, trifluoromethyl or nitro group, and the above described monosubstituted phenyl and naphthyl groups may also be substituted by a fluorine, chlorine or bromine atom, by a C₁₋₃-alkyl or C₁₋₃-alkoxy group and the above described disubstituted phenyl groups may also be substituted by a C₁₋₃-alkyl or C₁₋₃-alkoxy group, a naphthyl group, a chromane or chromene group in which a methylene group may be replaced by a carbonyl group, a 5- or 6-membered heteroaryl group which may be substituted in the carbon skeleton by a fluorine, chlorine or bromine atom, by a C₁₋₃-alkyl or C₁₋₃-alkoxy group, while the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms while the phenyl ring may also be substituted in the carbon skeleton by a fluorine, chlorine or bromine atom, by a C₁₋₃-alkyl or C₁₋₃-alkoxy group, a phenylvinyl group or R₁ together with A and the carbon atom between them are a C₅₋₇-cycloalkylidene group to which a phenyl ring may be fused by two adjacent carbon atoms, and the phenyl ring may also be substituted by one or two C₁₋₃-alkyl or C₁₋₃-alkoxy groups, while the substituents may be identical or different, and B is a 5- or 6-membered heteroaryl group substituted by a carboxy group or capable of being converted into a carboxy group in vivo, a phenyl or naphthyl group, each of which may be substituted by a carboxy group, by a group which may be converted into a carboxy group in vivo or by a group which is negatively charged under physiological conditions, and the above described phenyl groups may also be substituted by a fluorine, chlorine, bromine or iodine atom, by a C₁₋₃-alkyl, trifluoromethyl, phenyl, hydroxy, C₁₋₃-alkoxy, C₁₋₃-alkylsulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by a C₁₋₃-alkyl group which is substituted by a hydroxy, C₁₋₃-alkoxy, amino, C₁₋₄-alkylamino, di-(C₁₋₄-alkyl)-amino, C₃₋₇-cycloalkylamino, pyrrolidino, piperidino, morpholino, piperazino or N—(C₁₋₃-alkyl)-piperazino group, by an n-C₂₋₃-alkoxy, C₂₋₃-alkenyl or C₂₋₃-alkynyl group substituted in the 2 or 3 position by a di-(C₁₋₃-alkyl)-amino group, by an amino group, by an N—(C₁₋₃-alkyl)-amino or N,N-di-(C₁₋₃-alkyl)-amino group in which the alkyl moiety may each be substituted in the 2 or 3 position in relation to the nitrogen atom by a C₁₋₃-alkoxy group, by a N-phenylamino, N-(phenyl-C₁₋₃-alkyl)-amino or N-(pyridyl-C₁₋₃-alkyl)-amino group in which in each case a hydrogen atom of the above described amino groups may be substituted by a C₁₋₃-alkylsulphonyl, phenyl-C₁₋₃-alkylsulphonyl or phenylsulphonyl group or by a C₁₋₇-alkyl group, which may be substituted in the 2 to 5 position by a C₁₋₃-alkoxy, cyano, amino, C₁₋₃-alkylamino, di-(C₁₋₃-alkyl)-amino or tetrazolyl group, by an aminocarbonyl or C₁₋₃-alkylaminocarbonyl group which may in each case be substituted at the amino-nitrogen atom by a C₁₋₄-alkyl group which may be substituted by a vinyl, ethynyl, phenyl, pyridyl, imidazolyl, carboxy or trifluoromethyl group or, with the exception of the 2 position based on the aminocarbonyl nitrogen atom, by a hydroxy, C₁₋₃-alkoxy, C₁₋₃-alkylthio, amino, C₁₋₃-alkylamino, di-(C₁₋₃-alkyl)-amino, C₁₋₄-alkanoylamino or C₁₋₅-alkoxycarbonylamino group, by a C₃₋₇-cycloalkyl, C₅₋₉-Azabicycloalkyl, phenyl, pyridyl, C₁₋₃-alkoxy or di-(C₁₋₃-alkyl)-amino group, by a C₁₋₃-alkyl group which is substituted by a piperidin-3-yl or piperidin-4-yl group which may be substituted in the 1 position by a C₁₋₃-alkyl or C₁₋₅-alkoxycarbonyl group, or by an amino, C₁₋₃-alkylamino or phenyl-C₁₋₃-alkylamino group which may be substituted at the amino-nitrogen atom by a C₁₋₄-alkanoyl, C₁₋₅-alkoxycarbonyl, benzoyl, pyrrolidino, piperidino, morpholino or N—(C₁₋₃-alkyl)-piperazino group, by a carbonyl group substituted by a pyrrolidino, pyrrolino, piperidino, morpholino or N—(C₁₋₃-alkyl)-piperazino group, by a sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an amino or N—(C₁₋₃-alkyl)-amino group which is substituted in each case at the amino-nitrogen atom by an aminocarbonyl, C₁₋₃-alkylaminocarbonyl, phenyl-C₁₋₃-alkylaminocarbonyl, phenylaminocarbonyl, phenoxyphenylaminocarbonyl, pyridylaminocarbonyl, pyrrolidinocarbonyl, piperidinocarbonyl, morpholinocarbonyl or N—(C₁₋₃-alkyl)-piperazinocarbonyl group, and any hydrogen atom present in the above described aminocarbonyl groups may also be substituted by a C₁₋₃-alkyl group, by a 5- or 6-membered heteroaryl group, by a dihydro-oxazolyl, dihydro-imidazolyl, 2-oxo-pyrrolidino, 2-oxo-piperidino or 2-oxo-hexamethyleneimino group to which a phenyl ring may be fused by two adjacent carbon atoms, by an ethynyl group substituted by a phenyl, hydroxymethyl or dimethylamino group, and also the above described mono- or disubstituted phenyl groups may be substituted by another fluorine, chlorine or bromine atom or by one or two other C₁₋₃-alkyl or C₁₋₃-alkoxy groups and two C₁₋₃-alkoxy groups in the o position may be substituted by a methylenedioxy group, in particular R₁ indicates a hydrogen atom, a C₁₋₃-alkyl or trifluoromethyl group, R₂ indicates a hydrogen, fluorine, chlorine or bromine atom, a C₁₋₃-alkyl, C₃₋₇-cycloalkyl or C₁₋₃-alkoxy group or, if R₄ and R₅ each are a hydrogen atom, R₁ and R₂ together are an n-C₁₋₃-alkylene group which may be substituted by a C₁₋₃-alkyl group, R₃ is a hydrogen atom or a C₁₋₅-alkyl group, R₄ and R₅ each are a hydrogen atom or together are another carbon-carbon bond, A is a phenyl, naphthyl or tetrahydronaphthyl group substituted by a fluorine, chlorine, bromine or iodine atom, by a C₁₋₆-alkyl, C₃₋₇-cycloalkyl, phenyl, C₁₋₃-alkoxy, trifluoromethyl or nitro group, and the above described monosubstituted phenyl and naphthyl groups may also be substituted by a fluorine, chlorine or bromine atom, or by a C₁₋₃-alkyl or C₁₋₃-alkoxy group, a naphthyl group, a chromane or chromene group in which a methylene group may be replaced by a carbonyl group, a 5- or 6-membered heteroaryl group which may be substituted in the carbon skeleton by a fluorine, chlorine or bromine atom or by a C₁₋₃-alkyl or C₁₋₃-alkoxy group, and the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms and the phenyl ring may also be substituted in the carbon skeleton by a fluorine, chlorine or bromine atom or by a C₁₋₃-alkyl or C₁₋₃-alkoxy group, a phenylvinyl group or R₁ together with A and the carbon atom between them indicate a C₅₋₇-cycloalkylidene group to which a phenyl ring may be fused by two adjacent carbon atoms, and the phenyl ring may also be substituted by one or two C₁₋₃-alkyl or C₁₋₃-alkoxy groups, and the substituents may be identical or different, and B indicates a phenyl, naphthyl or heteroaryl group, each of which may be substituted by a carboxy group, by a group which may be converted into a carboxy group in vivo or by a group which is negatively charged under physiological conditions, and the above described phenyl groups may also be substituted by a fluorine, chlorine, bromine or iodine atom, by a C₁₋₃-alkyl, hydroxy, C₁₋₃-alkoxy, C₁₋₃-alkylsulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by an n-C₂₋₃-alkoxy group substituted in the 2 or 3 position by a di-(C₁₋₃-alkyl)-amino group, by an amino, N—(C₁₋₃-alkyl)-amino, N-(phenyl-C₁₋₃-alkyl)-amino or N-(pyridyl-C₁₋₃-alkyl)-amino group in which in each case a hydrogen atom of the amino group may be substituted by a C₁₋₃-alkylsulphonyl or phenylsulphonyl group or by a C₁₋₇-alkyl group, which may be substituted in the 2 to 5 position by a C₁₋₃-alkoxy, cyano, amino, C₁₋₃-alkylamino, di-(C₁₋₃-alkyl)-amino or tetrazolyl group, by a carbonyl or sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an imidazolyl or pyrazolyl group which may be substituted by a C₁₋₄-alkyl group, which may also be substituted by a C₁₋₃-alkyl, phenyl, trifluoromethyl or furyl group, and may also be substituted by another fluorine, chlorine or bromine atom, by another C₁₋₃-alkyl or C₁₋₃-alkoxy group, and the above described 6-membered heteroaryl groups contain one, two or three nitrogen atoms and the above described 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms, and the phenyl ring may be substituted in the carbon skeleton by a fluorine, chlorine or bromine atom or by a C₁₋₃-alkyl or C₁₋₃-alkoxy group, and the above described 5-membered monocyclic heteroaryl groups in the carbon skeleton may also be substituted by a C₁₋₄-alkyl, trifluoromethyl, phenyl or furanyl group and by another C₁₋₃-alkyl group, and amino and imino groups mentioned in the definition of the above described groups may also be substituted by a group which can be cleaved in vivo.

By a group which can be converted in vivo into a carboxy group is meant, for example, a hydroxmethyl group, a carboxy group esterified with an alcohol, in which the alcoholic moiety preferably indicates a C₁₋₆-alkanol, a phenyl-C₁₋₃-alkanol, a C₃₋₉-cycloalkanol, and a C₅₋₈-cycloalkanol may also be substituted by one or two C₁₋₃-alkyl groups, a C₅₋₈-cycloalkanol in which a methylene group in the 3 or 4 position is replaced by an oxygen atom or by an imino group which may be substituted by a C₁₋₃-alkyl, phenyl-C₁₋₃-alkyl, phenyl-C₁₋₃-alkoxycarbonyl or C₂₋₆-alkanoyl group and the cycloalkanol moiety may also be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-Cycloalkyl-C₁₋₃-alkanol, a bicycloalkanol having a total of 8 to 10 carbon atoms which may also be substituted by one or two C₁₋₃-alkyl groups in the bicycloalkyl moiety, a 1,3-dihydro-3-oxo-1-isobenzfuranol or an alcohol of formula I in which Rₐ indicates a C₁₋₈-alkyl, C₅₋₇-cycloalkyl, phenyl or phenyl-C₁₋₃-alkyl group, R_{b} indicates a hydrogen atom, a C₁₋₃-alkyl, C₅₋₇-cycloalkyl or phenyl group and R_{c} indicates a hydrogen atom or a C₁₋₃-alkyl group, by a group which is negatively charged under physiological conditions is meant a carboxy, hydroxysulphonyl, phosphono, tetrazol-5-yl, phenylcarbonylaminocarbonyl, trifluoromethylcarbonylaminocarbonyl, C₁₋₆-alkylsulphonyl-amino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group and by a group which can be cleaved in vivo from an imino or amino group is meant, for example, a hydroxy group, an acyl group such as the benzoyl or pyridinoyl group or a C₁₋₁₆-alkanoyl group such as the formyl, acetyl, propionyl, butanoyl, pentanoyl or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert. butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or Rₐ—CO—O—(R_{b} CR_{c})—O—CO group in which Rₐ to R_{c} are as defined above.

Moreover, the saturated alkyl and alkoxy moieties containing more than 2 carbon atoms mentioned in the definitions given above also include the branched isomers thereof, such as the isopropyl, tert.butyl, isobutyl group, etc.

Alternatively, in the above general formula I, R₁ indicates a hydrogen atom, a C₁₋₃-alkyl or trifluoromethyl group, R₂ indicates a hydrogen, fluorine, chlorine or bromine atom, a C₁₋₃-alkyl, C₃₋₇-cycloalkyl or C₁₋₃-alkoxy group or, if R₄ and R₅ each indicate a hydrogen atom, R₁ and R₂ together indicates a n-C₁₋₃-alkylene group which may be substituted by a C₁₋₃-alkyl group, R₃ indicates a hydrogen atom or a C₁₋₅-alkyl group, R₄ and R₅ each indicate a hydrogen atom or together indicate another carbon-carbon bond, A indicates a phenyl, naphthyl or tetrahydronaphthyl group substituted by a fluorine, chlorine, bromine or iodine atom, by a C₁₋₆-alkyl, C₃₋₇-cycloalkyl, phenyl, C₁₋₃-alkoxy, cyano, trifluoromethyl or nitro group, and the above described monosubstituted phenyl and naphthyl groups may also be substituted by a fluorine, chlorine or bromine atom, by a C₁₋₃-alkyl or C₁₋₃-alkoxy group and the above described disubstituted phenyl groups may also be substituted by a C₁₋₃-alkyl or C₁₋₃-alkoxy group, with the proviso that A does not indicate a phenyl group which is substituted by a halogen atom, by a methyl, pentyl, C₁₋₃-alkoxy or phenyl group or by two C₁₋₃-alkoxy groups, if R₃ indicates a hydrogen atom, R₄ and R₅ each indicate a hydrogen atom or R₄ and R₅ together indicate another carbon-carbon bond and B indicates a carboxyphenyl or methoxycarbonylphenyl group, and A does not indicate a phenyl group substituted by a methyl or phenyl group if R₁ and R₂ each indicate a hydrogen atom, R₃ indicates a hydrogen atom, R₄ and R₅ together indicate another carbon-carbon bond and B indicates a carboxyphenyl or methoxycarbonylphenyl group, a naphthyl group, a chromane or chromene group in which a methylene group may be replaced by a carbonyl group, a 5- or 6-membered heteroaryl group which may be substituted in the carbon skeleton by a fluorine, chlorine or bromine atom or by a C₁₋₃-alkyl or C₁₋₃-alkoxy group, and the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms, and the phenyl ring may also be substituted in the carbon skeleton by a fluorine, chlorine or bromine atom, by a C₁₋₃-alkyl or C₁₋₃-alkoxy group, a phenylvinyl group or R₁ together with A and the carbon atom between them indicate a C₅₋₇-cycloalkylidene group to which a phenyl ring may be fused by two adjacent carbon atoms, and the phenyl ring may also be substituted by one or two C₁₋₃-alkyl or C₁₋₃-alkoxy groups, and the substituents may be identical or different, and B indicates a 5- or 6-membered heteroaryl group substituted by a carboxy group or by a group which may be converted into a carboxy group in vivo, a phenyl or naphthyl group, each of which may be substituted by a carboxy group, by a group which may be converted into a carboxy group in vivo or by a group which is negatively charged under physiological conditions, and the above described phenyl groups may also be substituted by a fluorine, chlorine, bromine or iodine atom, by a C₁₋₃-alkyl, trifluoromethyl, phenyl, hydroxy, C₁₋₃-alkoxy, C₁₋₃-alkylsulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by a C₁₋₃-alkyl group which is substituted by a hydroxy, C₁₋₃-alkoxy, amino, C₁₋₄-alkylamino, di-(C₁₋₄-alkyl)-amino, C ₃₋₇-cycloalkylamino, pyrrolidino, piperidino, morpholino, piperazino or N—(C₁₋₃-alkyl)-piperazino group, by an n-C₂₋₃-alkoxy, C₂₋₃-alkenyl or C₂₋₃-alkynyl group substituted in the 2 or 3 position by a di-(C₁₋₃-alkyl)-amino group, by an amino group, by an N—(C₁₋₃-alkyl)-amino or N,N-di-(C₁₋₃-alkyl)-amino group in which the alkyl moiety may in each case be substituted in the 2 or 3 position in relation to the nitrogen atom by a C₁₋₃-alkoxy group, by an N-phenylamino, N-(phenyl-C₁₋₃-alkyl)-amino or N-(pyridyl-C₁₋₃-alkyl)-amino group in which in each case a hydrogen atom of the above described amino groups may be substituted by a C₁₋₃-alkylsulphonyl, phenyl-C₁₋₃-alkylsulphonyl or phenylsulphonyl group or by a C₁₋₇-alkyl group which may be replaced in the 2 to 5 position by a C₁₋₃-alkoxy, cyano, amino, C₁₋₃-alkylamino, di-(C₁₋₃-alkyl)-amino or tetrazolyl group, by an aminocarbonyl or C₁₋₃-alkylaminocarbonyl group which may in each case be substituted at the amino-nitrogen atom by a C₁₋₄-alkyl group which may be substituted by a vinyl, ethynyl, phenyl, pyridyl, imidazolyl, carboxy or trifluoromethyl group or, with the exception of the 2 position relative to the aminocarbonyl nitrogen atom, by a hydroxy, C₁₋₃-alkoxy, C₁₋₃-alkylthio, amino, C₁₋₃-alkylamino, di-(C₁₋₃-alkyl)-amino, C₁₋₄-alkanoylamino or C₁₋₅-alkoxycarbonylamino group, by a C₃₋₇-cycloalkyl, C₅₋₉-azabicycloalkyl, phenyl, pyridyl, C₁₋₃-alkoxy or di-(C₁₋₃-alkyl)-amino group, by a C₁₋₃-alkyl group which is substituted by a piperidin-3-yl or piperidin-4-yl group which may be substituted in the 1 position by a C₁₋₃-alkyl or C₁₋₅-alkoxycarbonyl group, or by an amino, C₁₋₃-alkylamino or phenyl-C₁₋₃-alkylamino group which may be substituted at the amino-nitrogen atom by a C₁₋₄-alkanoyl, C₁₋₅-alkoxycarbonyl, benzoyl, pyrrolidino, piperidino, morpholino or N—(C₁₋₃-alkyl)-piperazino group, by a carbonyl group substituted by a pyrrolidino, pyrrolino, piperidino, morpholino or N—(C₁₋₃-alkyl)-piperazino group, by a sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an amino or N—(C₁₋₃-alkyl)-amino group which may in each case be substituted at the amino-nitrogen atom by an aminocarbonyl, C₁₋₃-alkylaminocarbonyl, phenyl-C₁₋₃-alkylaminocarbonyl, phenylaminocarbonyl, phenoxyphenylaminocarbonyl, pyridylaminocarbonyl, pyrrolidinocarbonyl, piperidinocarbonyl, morpholinocarbonyl or N—(C₁₋₃-alkyl)-piperazinocarbonyl group, in which also any hydrogen atom of one of the above described aminocarbonyl groups present may be substituted by a C₁₋₃-alkyl group, by a 5- or 6-membered heteroaryl group, by a dihydro-oxazolyl, dihydro-imidazolyl, 2-oxo-pyrrolidino, 2-oxo-piperidino or 2-oxo-hexamethyleneimino group to which a phenyl ring may be fused by two adjacent carbon atoms, by an ethynyl group substituted by a phenyl, hydroxymethyl or dimethylamino group, and also the above described mono- or disubstituted phenyl groups may be substituted by another fluorine, chlorine or bromine atom or by one or two other C₁₋₃-alkyl or C₁₋₃-alkoxy groups and two C₁₋₃-alkoxy groups in the o position may be replaced by a methylenedioxy group, in particular R₁ indicates a hydrogen atom, a C₁₋₃-alkyl or trifluoromethyl group, R₂ indicates a hydrogen, fluorine, chlorine or bromine atom, a C₁₋₃-alkyl, C₃₋₇-cycloalkyl or C₁₋₃-alkoxy group or, if R₄ and R₅ each indicate a hydrogen atom, R₁ and R₂ together indicate an n-C₁₋₃-alkylene group which may be substituted by a C₁₋₃-alkyl group, R₃ indicates a hydrogen atom or a C₁₋₅-alkyl group, R₄ and R₅ each indicate a hydrogen atom or together indicate another carbon-carbon bond, A indicates a phenyl, naphthyl or tetrahydronaphthyl group substituted by a fluorine, chlorine, bromine or iodine atom or by a C₁₋₆-alkyl, C₃₋₇-cycloalkyl, phenyl, C₁₋₃-alkoxy, trifluoromethyl or nitro group, and the above described monosubstituted phenyl and naphthyl groups may also be substituted by a fluorine, chlorine or bromine atom or by a C₁₋₃-alkyl or C₁₋₃-alkoxy group, with the proviso that A does not indicate a phenyl group which is substituted by a halogen atom, by a methyl, pentyl, C₁₋₃-alkoxy or phenyl group or by two C₁₋₃-alkoxy groups if R₃ indicates a hydrogen atom, R₄ and R₅ each indicate a hydrogen atom or R₄ and R₅ together indicate another carbon-carbon bond and B indicates a carboxyphenyl or methoxycarbonylphenyl group, and A does not indicate a phenyl group which is substituted by a methyl or phenyl group if R₁ and R₂ each indicate a hydrogen atom, R₃ indicates a hydrogen atom, R₄ and R₅ together indicate another carbon-carbon bond and B indicates a carboxyphenyl or methoxycarbonylphenyl group, a naphthyl group, a chromane or chromene group in which a methylene group may be replaced by a carbonyl group, a 5- or 6-membered heteroaryl group which may be substituted in the carbon skeleton by a fluorine, chlorine or bromine atom or by a C₁₋₃-alkyl or C₁₋₃-alkoxy group, and the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms, and the phenyl ring may also be substituted in the carbon skeleton by a fluorine, chlorine or bromine atom, by a C₁₋₃-alkyl or C₁₋₃-alkoxy group, a phenylvinyl group or R₁ together with A and the carbon atom between them indicate a C₅₋₇-cycloalkylidene group to which a phenyl ring may be fused by two adjacent carbon atoms, and the phenyl ring may also be substituted by one or two C₁₋₃-alkyl or C₁₋₃-alkoxy group, and the substituents may be identical or different, and B indicates a phenyl, naphthyl or heteroaryl group, each of which may be substituted by a carboxy group, by a group which may be converted into a carboxy group in vivo or by a group which is negatively charged under physiological conditions, and the above described phenyl groups may also be substituted by a fluorine, chlorine, bromine or iodine atom, by a C₁₋₃-alkyl, hydroxy, C₁₋₃-alkoxy, C₁₋₃-alkylsulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by an n-C₂₋₃-alkoxy, C₂₋₃-alkenyl or C₂₋₃-alkynyl group substituted in the 2 or 3 position by a di-(C₁₋₃-alkyl)-amino group, by an amino, N—(C₁₋₃-alkyl)-amino, N-(phenyl-C₁₋₃-alkyl)-amino or N-(pyridyl-C₁₋₃-alkyl)-amino group in which in each case a hydrogen atom of the amino group may be substituted by a C₁₋₃-alkylsulphonyl or phenylsulphonyl group or by a C₁₋₇-alkyl group, which may be substituted in the 2 to 5 position by a C₁₋₃-alkoxy, cyano, amino, C₁₋₃-alkylamino, di-(C₁₋₃-alkyl)-amino or tetrazolyl group, by a carbonyl or sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an imidazolyl or pyrazolyl group which may be substituted by a C₁₋₄-alkyl group, which may also be substituted by a C₁₋₃-alkyl, phenyl, trifluoromethyl or furyl group, and may also be substituted by another fluorine, chlorine or bromine atom or by another C₁₋₃-alkyl or C₁₋₃-alkoxy group, and the above described 6-membered heteroaryl groups contain one, two or three nitrogen atoms and the above described 5-membered heteroaryl groups contain an imino group which may be substituted by a C₁₋₃-alkyl group, an oxygen or sulphur atom or an imino group which may be substituted by a C₁₋₃-alkyl group substituted and an oxygen or sulphur atom or one or two nitrogen atoms and also a phenyl ring may be fused to the above described monocyclic heteroaryl groups by two adjacent carbon atoms, this phenyl ring which may be being substituted in the carbon skeleton by a fluorine, chlorine or bromine atom or by a C₁₋₃-alkyl or C₁₋₃-alkoxy group, and the above described 5-membered monocyclic heteroaryl groups in the carbon skeleton may also be substituted by a C₁₋₄-alkyl, trifluoromethyl, phenyl or furanyl group and by another C₁₋₃-alkyl group, and the amino and imino groups mentioned in the definition of the above described groups may also be substituted by a group which may be cleaved in vivo, the isomers thereof and the salts thereof.

Other compounds of the above general formula I are those in which B and R₂ to R₅ are as hereinbefore defined, R₁ indicates a hydrogen atom or a C₁₋₃-alkyl group and A indicates a phenyl, naphthyl or tetrahydronaphthyl group substituted by a fluorine, chlorine, bromine or iodine atom or by a C₁₋₆-alkyl, C₃₋₇-cycloalkyl, phenyl, C₁₋₃-alkoxy, trifluoromethyl or nitro group, and the above described monosubstituted phenyl and naphthyl groups may also be substituted by a fluorine, chlorine or bromine atom or by a C₁₋₃-alkyl or C₁₋₃-alkoxy group, with the proviso that A does not indicate a phenyl group which may be mono- or disubstituted by halogen atoms, C₁₋₄-alkyl or C₁₋₃-alkoxy groups, in which the substituents may be identical or different, and does not represent a 4-biphenyl or pentylphenyl group if R₁ and R₂ each indicate a hydrogen atom or a C₁₋₄-alkyl group, R₃ indicates a hydrogen atom, R₄ and R₅ each indicate a hydrogen atom or R₄ and R₅ together indicate another carbon-carbon bond and B indicates a carboxyphenyl or methoxycarbonylphenyl group, a naphthyl group, a chromane or chromene group in which a methylene group may be replaced by a carbonyl group, a 5- or 6-membered heteroaryl group which may be substituted in the carbon skeleton by a fluorine, chlorine or bromine atom or by a C₁₋₃-alkyl or C₁₋₃-alkoxy group, and the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms, and the phenyl ring may also be substituted in the carbon skeleton by a fluorine, chlorine or bromine atom or by a C₁₋₃-alkyl or C₁₋₃-alkoxy group, the isomers thereof and the salts thereof.

Additional compounds of the above general formula I are those in which R₁ indicates a hydrogen atom or a C₁₋₃-alkyl group, R₂ indicates a hydrogen atom or a methyl group or, if R₄ and R₅ each indicate a hydrogen atom, R₁ and R₂ together indicate a methylene bridge, R₃ indicates a hydrogen atom or a C₁₋₅-alkyl group, R₄ and R₅ together indicate another carbon-carbon bond, A indicates a phenyl group substituted by a fluorine, chlorine, bromine or iodine atom or by a C₁₋₅-alkyl, cyclohexyl, phenyl, methoxy, cyano or trifluoromethyl group, a phenyl group substituted by fluorine, chlorine or bromine atoms, by methyl or methoxy groups, and the substituents may be identical or different, or a C₁₋₃-alkylphenyl group, which is disubstituted by fluorine, chlorine or bromine atoms, and the substituents may be identical or different, with the proviso that A does not indicate a phenyl group which is substituted by a halogen atom, by a methyl, pentyl, C₁₋₃-alkoxy or phenyl group or by two C₁₋₃-alkoxy groups, if R₃ indicates a hydrogen atom, R₄ and R₅ each indicate a hydrogen atom or R₄ and R₅ together indicate another carbon-carbon bond and B indicates a carboxyphenyl or methoxycarbonylphenyl group, and A does not indicate a phenyl group which is substituted by a methyl or phenyl group if R₁ and R₂ each indicate a hydrogen atom, R₃ indicates a hydrogen atom, R₄ and R₅ together indicate another carbon-carbon bond and B indicates a carboxyphenyl or methoxycarbonylphenyl group, a naphthyl group which may be substituted by a fluorine, chlorine or bromine atom or by a methyl or methoxy group, a tetrahydronaphthyl group, a chromene group in which a methylene group is replaced by a carbonyl group, a pyridyl, benzofuryl, benzothienyl, quinolyl or isoquinolyl group which may be substituted by a methyl group and B indicates a cyclohexyl, trimethoxyphenyl, methylenedioxyphenyl, naphthyl, pyridyl, thienyl, pyrazolyl, quinolyl or isoquinolyl group substituted by a carboxy group, a phenyl group substituted by a carboxy, methoxycarbonyl, ethoxycarbonyl, hydroxymethyl, sulpho, tetrazolyl, methylsulphonylaminocarbonyl or phenylsulphonylaminocarbonyl group, which may also be substituted by a fluorine, chlorine, bromine or iodine atom, by a methyl, trifluoromethyl, phenyl, hydroxymethyl, hydroxy, methoxy, methylsulphonyloxy, 2-dimethylamino-ethoxy, carboxy, nitro, methylsulphonylamino, phenylsulphonylamino, aminosulphonyl, pyrrolidino, piperidino or morpholino group, by a methyl group which is substituted by an amino, C₁₋₃-alkylamino, cyclopentylamino, pyrrolidino or piperidino group, by an amino, N-methyl-amino or N-(2-methoxy-ethyl)-amino group which may in each case be substituted at the amino-nitrogen atom by a C₁₋₇-alkyl or phenyl group, by an ethyl group which is substituted in the 1 or 2 position by a phenyl or pyridyl group, by a C₂₋₄-alkyl group which is terminally substituted by a methoxy, cyano, dimethylamino or tetrazolyl group, by an acetyl, benzoyl, C₁₋₅-alkoxycarbonyl, aminocarbonyl or methylaminocarbonyl group, and the aminocarbonyl moiety of the above described groups may in each case also be substituted by an which may be phenyl-substituted C₁₋₃-alkyl group, by a phenyl, phenoxyphenyl or pyridyl group, by a methylsulphonyl, phenylsulphonyl or benzylsulphonyl group, by an aminocarbonyl or methylaminocarbonyl group which may in each case be substituted at the amino-nitrogen atom by a C₁₋₄-alkyl, C₃₋₆-cycloalkyl, phenyl, benzyl, pyridyl, pyridylmethyl or methoxy group, by a methyl group which is substituted by a vinyl, ethynyl, trifluoromethyl, C₇₋₉-azabicycloalkyl, carboxy or imidazolyl group or by a piperidin-4-yl group which may be substituted in the 1 position by a methyl or C₁₋₅-alkoxycarbonyl group, by a straight-chain or branched C₂₋₃-alkyl group substituted in the 2 or 3 position by a hydroxy, methoxy, methylthio, amino, acetylamino, C₁₋₅-alkoxycarbonylamino, carboxy-, C₁₋₅-alkoxycarbonyl or dimethylamino group, by a pyrrolidino, piperidino, morpholino, 4-methyl-piperazino, amino or methylamino group, and the above described amino and methylamino groups may each also be substituted at the amino-nitrogen atom by a methyl, acetyl, benzoyl or C₁₋₅-alkoxycarbonyl group, by a dihydro-oxazolyl, dihydro-imidazolyl, 2-oxo-pyrrolidino, 2-oxo-piperidino or 2-oxo-hexamethyleneimino group to which a phenyl ring may be fused by two adjacent carbon atoms, by an imidazolyl or 4-methyl-imidazolyl group which may be substituted by a methyl, ethyl or phenyl group, to which a phenyl ring may also be fused by two adjacent carbon atoms, a pyrazolyl group which may be substituted by a C₁₋₄-alkyl or furanyl group, which may also be substituted by a methyl or trifluoromethyl group, by an ethynyl group substituted by a phenyl, hydroxymethyl or dimethylamino group, and also the above described mono- or disubstituted phenyl groups may be substituted by another fluorine, chlorine or bromine atom or by one or two other methyl or methoxy groups, particularly those compounds in which R₁ indicates a hydrogen atom or a C₁₋₃-alkyl group, R₂ indicates a hydrogen atom or R₁ and R₂ together indicate a methylene group, if R₄ and R₅ each simultaneously indicate a hydrogen atom, R₃ indicates a hydrogen atom, R₄ and R₅ together indicate another carbon-carbon bond, A indicates a phenyl or naphthyl group mono- or disubstituted by a fluorine, chlorine, bromine or iodine atom or by a C₁₋₆-alkyl, C₃₋₇-cycloalkyl or trifluoromethyl group, and the substituents may be identical or different, with the proviso that A does not indicate a phenyl group which may be mono- or di-substituted by halogen atoms or C₁₋₄-alkyl groups, in which the substituents may be identical or different, and does not indicate a 4-biphenyl or pentylphenyl group if R₁ indicates a hydrogen atom or a C₁₋₃-alkyl group, R₂ indicates a hydrogen atom, R₃ indicates a hydrogen atom, R₄ and R₅ each indicate a hydrogen atom or R₄ and R₅ together indicate another carbon-carbon bond and B indicates a carboxyphenyl or methoxycarbonylphenyl group, a naphthyl group, a chromene group in which a methylene group is replaced by a carbonyl group, a benzothienyl group and B indicates a phenyl, naphthyl, thienyl or pyridinyl group, each of which is substituted by a carboxy group, and the above described phenyl groups may also be substituted by a fluorine, chlorine or bromine atom, by a C₁₋₃-alkyl, hydroxy, C₁₋₃-alkoxy, C₁₋₃-alkylsulphonyloxy, pyrrolidino, piperidino, morpholino or N—(C₁₋₃-alkyl)-piperazino group, by an n-C₂₋₃-alkoxy group substituted in the 2 or 3 position by a di-(C₁₋₃-alkyl)-amino group, by an N-methyl-N-(n-C₂₋₃-alkyl)-amino group substituted in the 2 or 3 position by a di-(C₁₋₃-alkyl)-amino group, by a di-(C₁₋₃-alkyl)-amino group, by an imidazolyl or pyrazolyl group which may be substituted by a C₁₋₄-alkyl group, by a C₁₋₄-alkylaminocarbonyl, N-(pyridinylmethyl)-aminocarbonyl, pyrrolidinoaminocarbonyl or piperidinoaminocarbonyl group and may also be substituted by another fluorine atom, by another C₁₋₃-alkyl or C₁₋₃-alkoxy group, the isomers thereof and the salts thereof.

Further compounds of general formula I are those in which R₁ indicates a methyl group, R₂ indicates a hydrogen atom, R₃ indicates a hydrogen atom, R₄ and R₅ together indicate another carbon-carbon bond, A indicates a phenyl group substituted by two chlorine or bromine atoms or by a chlorine atom and a bromine atom, a naphthyl, 2-oxo-chromene or benzothienyl group, with the proviso that A does not indicate a phenyl group disubstituted by halogen atoms if R₁ indicates a methyl group, R₂ indicates a hydrogen atom, R₃ indicates a hydrogen atom, R₄ and R₅ each indicate a hydrogen atom or R₄ and R₅ together indicate another carbon-carbon bond and B indicates a carboxyphenyl or methoxycarbonylphenyl group, and B indicates a 2-carboxy-phenyl, 2-carboxy-thienyl or 2-carboxy-pyridinyl group, and the above described 2-carboxy-phenyl group may also be substituted in the phenyl nucleus by a fluorine, chlorine or bromine atom, by a C₁₋₃-alkyl, hydroxy, C₁₋₃-alkoxy, C₁₋₃-alkylsulphonyloxy or morpholino group, by an n-C₂₋₃-alkoxy group substituted in the 2 or 3 position by a di-(C₁₋₃-alkyl)-amino group, by an N-methyl-N-(n-C₂₋₃-alkyl)-amino group substituted in the 2 or 3 position by a di-(C₁₋₃-alkyl)-amino group, by an imidazolyl or pyrazolyl group which may be substituted by a C₁₋₄-alkyl group, by a C₁₋₄-alkylaminocarbonyl, N-(pyridinylmethyl)-aminocarbonyl, pyrrolidinoaminocarbonyl or piperidinoaminocarbonyl group and may also be substituted by another fluorine atom or by another methoxy group, the isomers thereof and the salts thereof.

Following are additional examples of carboxylic acid amide compounds: (1) trans-3-(naphth-2-yl)-but-2-enoic acid-N-(2-carboxy-phenyl)-amide, (2) trans-3-(naphth-2-yl)-but-2-enoic acid-N-(2-carboxy-4,5-dimethoxy-phenyl)-amide, (3) trans-3-(naphth-2-yl)-but-2-enoic acid-N-(2-carboxy-4-fluoro-phenyl)-amide, (4) trans-3-(naphth-2-yl)-but-2-enoic acid-N-(2-carboxy-4,5-difluoro-phenyl)-amide, (5) trans-3-(naphth-2-yl)-but-2-enoic acid-N-(2-carboxy-5-fluoro-phenyl)-amide, (6) trans-3-(naphth-2-yl)-but-2-enoic acid-N-(2-carboxy-4-methoxy-5-methyl-phenyl)-amide, (7) trans-3-(naphth-2-yl)-but-2-enoic acid-N-[2-carboxy-4-(morpholin-4-yl)-phenyl]-amide, (8) trans-3-(naphth-2-yl)-but-2-enoic acid-N-(2-carboxy-4-dimethylamino-phenyl)-amide, (9) trans-3-(naphth-2-yl)-but-2-enoic acid-N-(2-carboxy-4-hydroxy-phenyl)-amide, (10) trans-3-(naphth-2-yl)-but-2-enoic acid-N- (3-carboxy-thiophen-4-yl)-amide, (11) trans-3-(naphth-2-yl)-but-2-enoic acid-N-[2-carboxy-4-(imidazol-1-yl)-phenyl]-amide, (12) trans-3-(2-oxo-2H-chromen-yl)-but-2-enoic acid-N-(2-carboxy-phenyl)-amide, (13) trans-3-(naphth-2-yl)-but-2-enoic acid-N-[2-carboxy-4-(imidazol-1-yl)-5-fluoro-phenyl]-amide, (14) trans-3-(benzothiophen-2-yl)-but-2-enoic acid-N-(2-carboxy-phenyl)-amide, (15) trans-3-(naphth-2-yl)-but-2-enoic acid-N-(2-carboxy-4-methanesulphonyloxyphenyl)-amide, (16) trans-3-(naphth-2-yl)-but-2-enoic acid-N42-carboxy-4-(2-N,N-dimethylaminoethyloxy)-phenyl]-amide, (17) trans-3-(naphth-2-yl)-but-2-enoic acid-N-(4-carboxy-pyridin-3-yl)-amide, (18) trans-3-(3,4-dichlorphenyl)-but-2-enoic acid-N-(2-carboxy-4,5-dimethoxy-phenyl)-amide, (19) trans-3-(3-chloro-4-bromophenyl)-but-2-enoic acid-N-(2-carboxy-phenyl)-amide, (20) trans-3-(naphth-2-yl)-but-2-enoic acid-N-(2-carboxy-6-methyl-phenyl)-amide, (21) trans-3-(naphth-2-yl)-but-2-enoic acid-N-(2-carboxy-6-fluoro-phenyl)-amide, (22) trans-3-(naphth-2-yl)-but-2-enoic acid-N-[2-carboxy-5-(propylaminocarbonyl)-phenyl]-amide, (23) trans-3-(naphth-2-yl)-but-2-enoic acid-N-[2-carboxy-5-(pyrrolidin-1-yl-aminocarbonyl)-phenyl]-amide, (24) trans-3-(naphth-2-yl)-but-2-enoic acid-N-[2-carboxy-5-(N-(pyridin-3-yl-methyl)-aminocarbonyl)-phenyl]-amide, (25) trans-3-(naphth-2-yl)-but-2-enoic acid-N-(2-carboxy-6-chloro-phenyl)-amide and the isomers and salts thereof. Other exemplary compounds are the carboxylic acid amides selected from the group consisting of trans-4-bromocinnamic acid-N-(2-carboxyphenyl)-amide, trans-2-methylcinnamic acid-N-(2-carboxyphenyl)-amide, trans-4-methylcinnamic acid-N-(2-carboxyphenyl)-amide, trans-4-trifluoromethylcinnamic acid-N-(2-carboxyphenyl)-amide, trans-4-chlorocinnamic acid-N-(2-carboxyphenyl)-amide, trans-2-nitrocinnamic acid-N-(2-carboxyphenyl)-amide, trans-4-nitrocinnamic acid-N-(2-carboxyphenyl)-amide, trans-3-(furan-2-yl)prop-2-enoic acid-N-(2-carboxyphenyl)-amide, trans-3-(3′,4′dichlorophenyl)but-2-enoic acid-N-(2-carboxyphenyl)-amide, trans-3,4-dichlorocinnamic acid-N-(2-carboxyphenyl)-amide, trans-3-(biphenyl-4-yl)but-2-enoic acid-N-(2-carboxyphenyl)-amide, trans-3-(naphtha-2-yl)prop-2-enoic acid-N-(2-carboxyphenyl)-amide, trans-cinnamic acid-N-(2-carboxyphenyl)-amide, trans-3-(phenyl)pent-2-enoic acid-N-(2-carboxyphenyl)-amide, trans-4-methoxycinnamic acid-N-(2-carboxyphenyl)-amide, trans-3-(naphtha-2-yl)prop-2-enoic acid-N-methyl-N-(2-carboxyphenyl)-amide and trans-3-methoxy-4-benzoxycinnamic acid-N-(2-carboxyphenyl)-amide, and the isomers and salts thereof. In another embodiment, the carboxylic acid amide of the present invention is a carboxylic acid amide, trans-5-bromo-2-[[(2E)-3-(2-naphthalenyl)-1-oxo-2-butenyl]amino]benzoic acid.

Additional carboxylic acid amides that could be used in the method of the present invention include carboxylic acid amides of general formula II

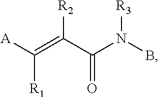

the isomers thereof, the trans-isomers thereof, and the salts thereof. In the carboxylic acid amides of the above general formula II, R₁ indicates a hydrogen atom, a C₁₋₃-alkyl or trifluoromethyl group, R₂ indicates a hydrogen, fluorine, chlorine or bromine atom or a C₁₋₃-alkyl group, R₃ indicates a hydrogen atom or a C₁₋₅-alkyl group, A indicates a phenyl or naphthyl group substituted by a fluorine, chlorine, bromine or iodine atom, by a C₁₋₆-alkyl, C₃₋₇-cycloalkyl, phenyl, C₁₋₃-alkoxy, cyano, trifluoromethyl or nitro group, and the above described monosubstituted phenyl and naphthyl groups may also be substituted by a fluorine, chlorine or bromine atom, by a C₁₋₃-alkyl or C₁₋₃-alkoxy group and the above described disubstituted phenyl groups may also be substituted by a C₁₋₃-alkyl or C₁₋₃-alkoxy group, a naphthyl group, a chromane or chromene group in which a methylene group may be replaced by a carbonyl group, or a 5- or 6-membered heteroaryl group which may be substituted in the carbon skeleton by a fluorine, chlorine or bromine atom or by a C₁₋₃-alkyl or C₁₋₃-alkoxy group, while the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom, or one or two nitrogen atoms and also a phenyl ring may be fused to the above described monocyclic heteroaryl groups by two adjacent carbon atoms and may also be substituted in the carbon skeleton by a fluorine, chlorine or bromine atom or by a C₁₋₃-alkyl or C₁₋₃-alkoxy group, and B indicates a phenyl or naphthyl group which is substituted in each case by a carboxy group, by a group which may be converted into a carboxy group in vivo or by a group which is negatively charged under physiological conditions and may which may be also be substituted by a fluorine, chlorine, bromine or iodine atom or by a C₁₋₃-alkyl, trifluoromethyl or methoxy group, and the above described phenyl groups are also substituted by a C₁₋₃-alkyl group which is substituted by an amino, C₁₋₄-alkylamino, di-(C₁₋₄-alkyl)-amino, C₃₋₇-cycloalkylamino, pyrrolidino, piperidino, morpholino, piperazino or N—(C₁₋₃-alkyl)-piperazino group, while the amino and imino groups mentioned in the definition of the above described groups may also be substituted by a group which can be cleaved in vivo.

By a group which can be converted in vivo into a carboxy group is meant, for example, a hydroxmethyl group, a carboxy group esterified with an alcohol, in which the alcoholic moiety preferably indicates a C₁₋₆-alkanol, a phenyl-C₁₋₃-alkanol, a C₃₋₉-cycloalkanol, and a C₅₋₈-cycloalkanol may also be substituted by one or two C₁₋₃-alkyl groups, a C₅₋₈-cycloalkanol in which a methylene group in the 3 or 4 position is replaced by an oxygen atom or by an imino group which may be substituted by a C₁₋₃-alkyl, phenyl-C₁₋₃-alkyl, phenyl-C₁₋₃-alkoxycarbonyl or C₂₋₆-alkanoyl group and the cycloalkanol moiety may also be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol having a total of 8 to 10 carbon atoms which may also be substituted by one or two C₁₋₃-alkyl groups in the bicycloalkyl moiety, a 1,3-dihydro-3-oxo-1-isobenzfuranol or an alcohol of Rₐ—CO—O—(R_{b} CR_{c})—O—OH in which Rₐ indicates a C₁₋₈-alkyl, C₅₋₇-cycloalkyl, phenyl or phenyl-C₁₋₃-alkyl group, R_{b} indicates a hydrogen atom, a C₁₋₃-alkyl, C₅₋₇-cycloalkyl or a phenyl group and R_{c} indicates a hydrogen atom or a C₁₋₃-alkyl group, by a group which is negatively charged under physiological conditions is meant a carboxy, hydroxysulphonyl, phosphono, tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group, and by a group which can be cleaved in vivo from an imino or amino group is meant, for example, a hydroxy group, an acyl group such as the benzoyl or pyridinoyl group or a C₁₋₆-alkanoyl group such as the formyl, acetyl, propionyl, butanoyl, pentanoyl or hexanoyl group, an allyloxycarbonyl group, a C₁₋₁₆-alkoxycarbonyl group such as the methoxycarbonyl, ethoxycarbonyl, propoxycarbonyl, isopropoxycarbonyl, butoxycarbonyl, tert. butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulphonyl-C₂₋₄-alkoxycarbonyl, C₁₋₃-alkoxy-C₂₋₄-alkoxy-C₂₋₄-alkoxycarbonyl or Rₐ—CO—O—(R_{b} CR_{c})—O—CO— group in which Rₐ to R_{c} are as hereinbefore defined with respect to formula II.

Moreover, the saturated alkyl and alkoxy moieties containing more than 2 carbon atoms mentioned in the definitions given above and hereinafter also include the branched isomers thereof, such as the isopropyl, tert.butyl, isobutyl group, etc.

Other compounds of the above general formula II are those in which R₁ indicates a hydrogen atom or a C₁₋₃-alkyl group, R₂ indicates a hydrogen, fluorine, chlorine or bromine atom or a C₁₋₃-alkyl group, R₃ indicates a hydrogen atom or a methyl group, A indicates a phenyl or naphthyl group substituted by a fluorine, chlorine, bromine or iodine atom, by a C₁₋₆-alkyl or C₁₋₃-alkoxy group which may also be substituted in each case by a fluorine, chlorine or bromine atom, by a C₁₋₃-alkyl or C₁₋₃-alkoxy group, a naphthyl group, a chromane or chromene group in which a methylene group may be replaced by a carbonyl group, or a 5 or 6-membered heteroaryl group, while the 6-membered heteroaryl groups contain one, two or three nitrogen atoms and the 5-membered heteroaryl groups contain an imino group which may be substituted by a C₁₋₃-alkyl group, an oxygen or sulphur atom, or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms, and B indicates a phenyl or naphthyl group which may be substituted in each case by a carboxy group, by a group which may be converted into a carboxy group in vivo or by a group which is negatively charged under physiological conditions and which may be also substituted by a fluorine, chlorine, bromine or iodine atom or by a C₁₋₃-alkyl, trifluoromethyl or methoxy group, and the above described phenyl groups are also substituted by a C₁₋₃-alkyl group, which is substituted by an amino, C₁₋₄-alkylamino, di-(C₁₋₄-alkyl)-amino, C₃₋₇-cycloalkylamino, pyrrolidino, piperidino, morpholino, piperazino or N—(C₁₋₃-alkyl)-piperazino group, the isomers thereof and the salts thereof.

Additional compounds of the above general formula II are those in which R₁ indicates a hydrogen atom or a C₁₋₃-alkyl group, R₂ indicates a hydrogen atom or a methyl group, R₃ indicates a hydrogen atom, A indicates a phenyl group mono- or disubstituted by fluorine, chlorine, bromine or iodine atoms, by C₁₋₅-alkyl or methoxy groups, while the substituents may be identical or different, or a naphthyl group which may be substituted by a fluorine, chlorine or bromine atom, by a methyl or methoxy group, a chromene group in which a methylene group is replaced by a carbonyl group, or a benzofuryl, benzothienyl, quinolyl or isoquinolyl group which may be substituted by a methyl group and B indicates a naphthyl group substituted by a carboxy group or a phenyl group substituted by a carboxy, methoxycarbonyl, ethoxycarbonyl or tetrazolyl group which may which may be be substituted by a fluorine, chlorine, bromine or iodine atom or by a C₁₋₃-alkyl, a trifluoromethyl or a methoxy group and is also substituted by a C₁₋₃-alkyl group, which is substituted by an amino, C₁₋₄-alkylamino, di-(C₁₋₄-alkyl)-amino, C₃₋₇-cycloalkylamino, pyrrolidino, piperidino, morpholino, piperazino or N—(C₁₋₃-alkyl)-piperazino group, the isomers thereof and the salts thereof.

Further compounds of the above general formula II are those in which R₁, R₂, R₃ and A are as hereinbefore defined, and B has the meanings given hereinbefore, while the carboxy, methoxycarbonyl, ethoxycarbonyl or tetrazolyl substituent is in the 2 position and the alkyl group which is substituted as described above is in the 5 position of the phenyl ring, the isomers thereof and the salts thereof, but particularly those compounds of general formula I in which R₁ indicates a methyl group, R₂ indicates a hydrogen atom, R₃ indicates a hydrogen atom, A indicates a naphthyl group and B indicates a 2-carboxy-phenyl group, while the above described 2-carboxy-phenyl group is also substituted in the phenyl nucleus in the 5 position by a methyl group which is substituted by an amino, C₁₋₄-alkylamino, di-(C₁₋₃-alkyl)-amino, cyclopentylamino or pyrrolidino group, the isomers thereof and the salts thereof.

The following are examples of carboxylic acid amide compounds of formula II: (1) trans-3-(naphth-2-yl)-but-2-enoic acid-N-(2-carboxy-5-dimethylaminomethyl-phenyl)-amide, (2) trans-3-(naphth-2-yl)-but-2-enoic acid-N-[2-carboxy-5-(pyrrolidin-1-yl)methyl-phenyl]-amide, (3) trans-3-(naphth-2-yl)-but-2-enoic acid-N-(2-carboxy-5-ethylaminomethyl-phenyl)-amide, (4) trans-3-(naphth-2-yl)-but-2-enoic acid-N-(2-carboxy-5-isopropylaminomethyl-phenyl)-amide, (5) trans-3-(naphth-2-yl)-but-2-enoic acid-N-(2-carboxy-5-cyclopentylaminomethyl-phenyl)-amide, and the isomers and salts thereof. Other exemplary compounds are the carboxylic acid amides selected from the group consisting of trans-3-(naphtha-2-yl)but-2-enoic acid-N-(2-carboxyphenyl)-amide, trans-4-bromocinnamic acid-N-(2-carboxyphenyl)-amide, trans-2-methylcinnamic acid-N-(2-carboxyphenyl)-amide, trans-4-methylcinnamic acid-N-(2-carboxyphenyl)-amide, trans-4-trifluoromethylcinnamic acid-N-(2-carboxyphenyl)-amide, trans-4-chlorocinnamic acid-N-(2-carboxyphenyl)-amide, trans-2-nitrocinnamic acid-N-(2-carboxyphenyl)-amide, trans-4-nitrocinnamic acid-N-(2-carboxyphenyl)-amide, trans-3-(furan-2-yl)prop-2-enoic acid-N-(2-carboxyphenyl)-amide, trans-3-(3′,4′dichlorophenyl)but-2-enoic acid-N-(2-carboxyphenyl)-amide, trans-3,4-dichlorocinnamic acid-N-(2-carboxyphenyl)-amide, trans-3-(biphenyl-4-yl)but-2-enoic acid-N-(2-carboxyphenyl)-amide, trans-3-(naphtha-2-yl)prop-2-enoic acid-N-(2-carboxyphenyl)-amide, trans-cinnamic acid-N-(2-carboxyphenyl)-amide, trans-3-(phenyl)pent-2-enoic acid-N-(2-carboxyphenyl)-amide, trans-4-methoxycinnamic acid-N-(2-carboxyphenyl)-amide, trans-3-(naphtha-2-yl)prop-2-enoic acid-N-methyl-N-(2-carboxyphenyl)-amide and trans-3-methoxy-4-benzoxycinnamic acid-N-(2-carboxyphenyl)-amide, and the isomers and salts thereof. In another embodiment, the carboxylic acid amide of the present invention is a carboxylic acid amide, trans-5-bromo-2-[[(2E)-3-(2-naphthalenyl)-1-oxo-2-butenyl]amino]benzoic acid.

Alternatively, in the carboxylic acid amides of the above general formula II, R₁ indicates a hydrogen atom or a C₁₋₃-alkyl group, R₂ indicates a hydrogen, fluorine, chlorine or bromine atom or a C₁₋₃-alkyl group, R₃ indicates a hydrogen atom or a C₁₋₅-alkyl group, A indicates a chromane or chromene group linked by a fused-on phenyl ring in which a methylene group may be replaced by a carbonyl group, or a bicyclic heteroaryl group consisting of a 5- or 6-membered heteroaryl group which may be substituted in the carbon skeleton by a fluorine, chlorine or bromine atom or by a C₁₋₃-alkyl or C₁₋₃-alkoxy group, in which the 6-membered heteroaryl groups contain one, two or three nitrogen atoms and the 5-membered heteroaryl groups contain an imino group which may be substituted by a C₁₋₃-alkyl group, an oxygen or sulphur atom, or an imino group which may be substituted by a C₁₋₃-alkyl group and an oxygen or sulphur atom or one or two nitrogen atoms, and a phenyl ring fused to the above described monocyclic heteroaryl groups by two adjacent carbon atoms, by means of which the bicyclic heteroaryl group is linked to the R₁-substituted alkene-carbon atom and which may also be substituted in the carbon skeleton by a fluorine, chlorine or bromine atom or by a C₁₋₃-alkyl or C₁₋₃-alkoxy group, and B indicates a 5- or 6-membered heteroaryl group substituted by a carboxy group or by a group which may be converted into a carboxy group in vivo or a phenyl or naphthyl group which is substituted in each case by a carboxy group, by a group which may be converted into a carboxy group in vivo or by a group which is negatively charged under physiological conditions, while the above described phenyl group may also be substituted by a fluorine, chlorine, bromine or iodine atom, by a C₁₋₃-alkyl, trifluoromethyl, phenyl, hydroxy, C₁₋₃-alkoxy, C₁₋₃-alkyl-sulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl- or di-(C₁₋₃-alkyl)-aminosulphonyl group, by an n-C₂₋₃-alkoxy group substituted in the 2 or 3 position by a di-(C₁₋₃-alkyl)-amino group, by an amino group, by an N—(C₁₋₃-alkyl)-amino or N,N-di-(C₁₋₃-alkyl)-amino group in which the alkyl moiety in the 2 or 3 position relative to the nitrogen atom may be substituted in each case by a C₁₋₃-alkoxy group, by an N-phenylamino, N-(phenyl-C₁₋₃-alkyl)-amino or N-(pyridyl-C₁₋₃-alkyl)-amino group, by an aminocarbonyl group which may be mono- or disubstituted at the amino-nitrogen atom by a C₁₋₃-alkyl group, by a pyrrolidinocarbonyl, piperidinocarbonyl, morpholinocarbonyl or N—(C₁₋₃-alkyl)-piperazinocarbonyl group, by a sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an amino or N—(C₁₋₃-alkyl)-amino group which is substituted in each case at the amino-nitrogen atom by an aminocarbonyl, C₁₋₃-alkylaminocarbonyl, phenyl-C₁₋₃-alkylaminocarbonyl, phenylaminocarbonyl, pyridylaminocarbonyl, pyrrolidinocarbonyl, piperidinocarbonyl, morpholinocarbonyl or N—(C₁₋₃-alkyl)-piperazinocarbonyl group, and in the above described aminocarbonyl groups any hydrogen atom present may also be replaced by a C₁₋₃-alkyl group, or by a 5 or 6-membered heteroaryl group, and the above described phenyl groups may also be substituted by another fluorine, chlorine or bromine atom or by another C₁₋₃-alkyl or C₁₋₃-alkoxy group and two C₁₋₃-alkoxy groups in the o position may be replaced by a methylenedioxy group, and the above described 6-membered heteroaryl groups contain one, two or three nitrogen atoms and the above described 5-membered heteroaryl groups contain an imino group which may be substituted by a C₁₋₃-alkyl group, an oxygen or sulphur atom, or an imino group which may be substituted by a C₁₋₃-alkyl group and an oxygen or sulphur atom or one or two nitrogen atoms, the isomers thereof and the salts thereof.

Of note: A indicates a bicyclic heteroaryl group which is linked by the phenyl ring to the R₁-substituted olefinic carbon atom.

By a group which can be converted in vivo into a carboxy group is meant, for example, a hydroxmethyl group, a carboxy group esterified with an alcohol, in which the alcoholic moiety preferably indicates a C₁₋₆-alkanol, a phenyl-C₁₋₃-alkanol, a C₃₋₉-cycloalkanol, and a C₅₋₈-cycloalkanol may also be substituted by one or two C₁₋₃-alkyl groups, a C₅₋₈-cycloalkanol in which a methylene group in the 3 or 4 position is replaced by an oxygen atom or by an imino group which may be substituted by a C₁₋₃-alkyl, phenyl-C₁₋₃-alkyl, phenyl-C₁₋₃-alkoxycarbonyl or C₂₋₆-alkanoyl group and the cycloalkanol moiety may also be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol, with the proviso that no bond to the oxygen atom starts from a carbon atom which carries a double or triple bond, a C₃₋₈-cycloalkyl-C₁₋₃-alkanol, a bicycloalkanol having a total of 8 to 10 carbon atoms which may also be substituted by one or two C₁₋₃-alkyl groups in the bicycloalkyl moiety, a 1,3-dihydro-3-oxo-1-isobenzfuranol or an alcohol of formula Rₐ—CO—O—(R_{b} CR_{c})—O—OH in which Rₐ indicates a C₁₋₈-alkyl, C₅₋₇-cycloalkyl, phenyl or phenyl-C₁₋₃-alkyl group, R_{b} indicates a hydrogen atom, a C₁₋₃-alkyl, C₅₋₇-cycloalkyl or phenyl group and R_{c} indicates a hydrogen atom or a C₁₋₃-alkyl group, by a group which is negatively charged under physiological conditions is meant a carboxy, hydroxysulphonyl, phosphono, tetrazol-5-yl, phenylcarbonylaminocarbonyl, trifluoromethylcarbonylaminocarbonyl, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group.

Moreover, the saturated alkyl and alkoxy moieties containing more than 2 carbon atoms mentioned in the definitions given above and hereinafter also include the branched isomers thereof, such as the isopropyl, tert.butyl, isobutyl group, etc.

Additional compounds of the above general formula II are those in which R₁ indicates a hydrogen atom or a C₁₋₃-alkyl group, R₂ indicates a hydrogen, fluorine, chlorine or bromine atom or a C₁₋₃-alkyl group, R₃ indicates a hydrogen atom or a methyl group, A indicates a chromane or chromene group linked by a fused-on phenyl ring in which a methylene group may be replaced by a carbonyl group, or a bicyclic heteroaryl group consisting of a 5 or 6-membered heteroaryl group which may be substituted in the carbon skeleton by a fluorine, chlorine or bromine atom, by a C₁₋₃-alkyl or C₁₋₃-alkoxy group, while the 6-membered heteroaryl groups contain one, two or three nitrogen atoms and the 5-membered heteroaryl groups contain an imino group which may be substituted by a C₁₋₃-alkyl group, an oxygen or sulphur atom, or an imino group which may be substituted by a C₁₋₃-alkyl group and an oxygen or sulphur atom or one or two nitrogen atoms, and a phenyl ring fused to the above described monocyclic heteroaryl groups by two adjacent carbon atoms, by means of which the bicyclic heteroaryl group is linked to the R₁-substituted alkene-carbon atom and which may also be substituted in the carbon skeleton by a fluorine, chlorine or bromine atom or by a methyl or methoxy group, and B indicates a 5 or 6-membered heteroaryl group substituted by a carboxy group or by a group which may be converted into a carboxy group in vivo or a phenyl or naphthyl group which is substituted in each case by a carboxy group or by a group which may be converted into a carboxy group in vivo, while the above described phenyl group is also substituted by a fluorine, chlorine or bromine atom, by a C₁₋₃-alkyl, trifluoromethyl, phenyl, hydroxy, C₁₋₃-alkoxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by a n-C₂₋₃-alkoxy group substituted in the 2 or 3 position by a di-(C₁₋₃-alkyl)-amino group, by an amino group, by an N—(C₁₋₃-alkyl)-amino or N,N-di-(C₁₋₃-alkyl)-amino group in which the alkyl moiety in the 2 or 3 position relative to the nitrogen atom may be substituted in each case by a C₁₋₃-alkoxy group, by an N-phenylamino, N-(phenyl-C₁₋₃-alkyl)-amino or N-(pyridyl-C₁₋₃-alkyl)-amino group, by an aminocarbonyl group which may be mono or disubstituted at the amino-nitrogen atom by a C₁₋₃-alkyl group, by a pyrrolidinocarbonyl, piperidinocarbonyl, morpholinocarbonyl or N—(C₁₋₃-alkyl)-piperazino carbonyl group, by a sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, or by an amino or N—(C₁₋₃-alkyl)-amino group which is substituted in each case at the amino-nitrogen atom by an aminocarbonyl, C₁₋₃-alkylaminocarbonyl, phenyl-C₁₋₃-alkylamino carbonyl, phenylamino carbonyl, pyridylaminocarbonyl, pyrrolidinocarbonyl, piperidinocarbonyl, morpholinocarbonyl or N—(C₁₋₃-alkyl)-piperazino carbonyl group, in which also any hydrogen atom present in one of the above described aminocarbonyl groups may be replaced by a C₁₋₃-alkyl group, and the above described phenyl groups may also be substituted by another fluorine, chlorine or bromine atom or by another C₁₋₃-alkyl or C₁₋₃-alkoxy group, and the above described 6-membered heteroaryl groups contain one or two nitrogen atoms and the above described 5-membered heteroaryl groups contain an imino group which may be substituted by a C₁₋₃-alkyl group, an oxygen or sulphur atom, or an imino group which may be substituted by a C₁₋₃-alkyl group and an oxygen or sulphur atom or one or two nitrogen atoms, the isomers thereof and the salts thereof.

Some particular compounds of the above general formula I are those in which B and R₁ to R₃ are as hereinbefore defined, and A indicates a chromane or chromene group linked by a fused-on phenyl ring in which a methylene group may be replaced by a carbonyl group, or a bicyclic heteroaryl group consisting of a 5 or 6-membered heteroaryl group which may be substituted in the carbon skeleton by a fluorine, chlorine or bromine atom, by a methyl or methoxy group, while the 6-membered heteroaryl groups contain one, two or three nitrogen atoms and the 5-membered heteroaryl groups contain an imino group which may be substituted by a methyl group, an oxygen or sulphur atom, or an imino group which may be substituted by a methyl group and an oxygen or sulphur atom or one or two nitrogen atoms, and a phenyl ring fused to the above described monocyclic heteroaryl groups by two adjacent carbon atoms, by means of which the bicyclic heteroaryl group is linked to the R₁ substituted alkene-carbon atom and which may also be substituted in the carbon skeleton by a fluorine, chlorine or bromine atom or by a methyl or methoxy group, the isomers thereof and the salts thereof.

Further, compounds of general formula II are those in which R₁ indicates a hydrogen atom or a C₁₋₃-alkyl group, R₂ indicates a hydrogen atom or a methyl group, R₃ indicates a hydrogen atom, A indicates a benzofuryl, benzothienyl, quinolyl or isoquinolyl group bound by the phenyl moiety to the R₁-substituted alkene-carbon atom and which may be substituted by a methyl group and B indicates a pyridyl, thienyl, pyrazolyl, quinolyl or isoquinolyl group substituted by a carboxy group or a phenyl group substituted by a carboxy, methoxycarbonyl or ethoxycarbonyl group, which may also be substituted by a fluorine, chlorine or bromine atom, by a methyl, trifluoromethyl, phenyl, hydroxymethyl, hydroxy, methoxy, 2-dimethylamino-ethoxy, nitro, methylsulphonylamino, phenylsulphonylamino, aminosulphonyl, pyrrolidino, piperidino or morpholino group, by an amino, N-methyl-amino or N-(2-methoxy-ethyl)-amino group which may be substituted in each case at the amino-nitrogen atom by a C₁₋₃-alkyl group, or by an aminocarbonyl, methylaminocarbonyl or dimethylaminocarbonyl group, and the above described phenyl groups may also be substituted by another fluorine or chlorine atom or by another methyl or methoxy group, the isomers thereof and the salts thereof, particularly those compounds of general formula II in which R₁ indicates a hydrogen atom, a methyl or ethyl group, R₂ indicates a hydrogen atom, R₃ indicates a hydrogen atom, A indicates a benzothienyl, quinolyl or isoquinolyl group bound to the R₁-substituted alkene-carbon atom by the phenyl moiety and which may be substituted by a methyl group and B indicates a phenyl, thienyl or pyridinyl group substituted in each case by a carboxy group, while the above described phenyl group may also be substituted by a fluorine, chlorine or bromine atom, by a methyl, hydroxy, C₁₋₃-alkoxy, pyrrolidino, piperidino, morpholino or N—(C₁₋₃-alkyl)-piperazino group, by a 2-dimethylaminoethoxy group, by an amino, methylamino or dimethylamino group or by an aminocarbonyl, methylaminocarbonyl or dimethylaminocarbonyl group and the disubstituted phenyl groups may also be substituted by another fluorine atom or by another methyl or methoxy group, the isomers thereof and the salts thereof.

With other compounds of general formula II, R₁ indicates a methyl group, R₂ indicates a hydrogen atom, R₃ indicates a hydrogen atom, A indicates a benzothienyl, quinolyl or isoquinolyl group bound to the R₁-substituted alkene-carbon atom by the phenyl moiety and B indicates a 2-carboxy-phenyl or 2-carboxy-thienyl group, while the above described 2-carboxy-phenyl group may also be substituted in the phenyl nucleus by a fluorine, chlorine or bromine atom, by a methyl, hydroxy, C₁₋₃-alkoxy, amino, methylamino, dimethylamino or morpholino group, by a 2-dimethylaminoethoxy group or by an aminocarbonyl, methylaminocarbonyl or dimethylaminocarbonyl group and which may be also by another fluorine atom or by another methoxy group, the isomers thereof and the salts thereof.

The following carboxylic acid amides are exemplary compounds: (1) trans-3-(benzothien-6-yl)-but-2-enoic acid-N-(2-carboxy-phenyl)-amide, (2) trans-3-(benzothien-5-yl)-but-2-enoic acid-N-(2-carboxy-phenyl)-amide, (3) trans-3-(benzothien-6-yl)-but-2-enoic acid-N-(2-carboxy-4,5-dimethoxyphenyl)-amide, (4) trans-3-(benzothien-6-yl)-but-2-enoic acid-N-(2-carboxy-6-methylphenyl)-amide, (5) trans-3-(benzothien-6-yl)-but-2-enoic acid-N-(2-carboxy-4-fluorophenyl)-amide and (6) trans-3-(quinolin-6-yl)-but-2-enoic acid-N-(2-carboxy-phenyl)-amide, as well as the isomers and salts thereof. Other exemplary compounds are the carboxylic acid amides selected from the group consisting of trans-3-(naphtha-2-yl)but-2-enoic acid-N-(2-carboxyphenyl)-amide, trans-4-bromocinnamic acid-N-(2-carboxyphenyl)-amide, trans-2-methylcinnamic acid-N-(2-carboxyphenyl)-amide, trans-4-methylcinnamic acid-N-(2-carboxyphenyl)-amide, trans-4-trifluoromethylcinnamic acid-N-(2-carboxyphenyl)-amide, trans-4-chlorocinnamic acid-N-(2-carboxyphenyl)-amide, trans-2-nitrocinnamic acid-N-(2-carboxyphenyl)-amide, trans-4-nitrocinnamic acid-N-(2-carboxyphenyl)-amide, trans-3-(furan-2-yl)prop-2-enoic acid-N-(2-carboxyphenyl)-amide, trans-3-(3′,4′dichlorophenyl)but-2-enoic acid-N-(2-carboxyphenyl)-amide, trans-3,4-dichlorocinnamic acid-N-(2-carboxyphenyl)-amide, trans-3-(biphenyl-4-yl)but-2-enoic acid-N-(2-carboxyphenyl)-amide, trans-3-(naphtha-2-yl)prop-2-enoic acid-N-(2-carboxyphenyl)-amide, trans-cinnamic acid-N-(2-carboxyphenyl)-amide, trans-3-(phenyl)pent-2-enoic acid-N-(2-carboxyphenyl)-amide, trans-4-methoxycinnamic acid-N-(2-carboxyphenyl)-amide, trans-3-(naphtha-2-yl)prop-2-enoic acid-N-methyl-N-(2-carboxyphenyl)-amide and trans-3-methoxy-4-benzoxycinnamic acid-N-(2-carboxyphenyl)-amide, and the isomers and salts thereof.

In another embodiment, the carboxylic acid amide of the present invention is a carboxylic acid amide, trans-5-bromo-2-[[(2E)-3-(2-naphthalenyl)-1-oxo-2-butenyl]amino]benzoic acid.

The carboxylic acid amides of the present invention include, isomers, trans-isomers, and salts of carboxylic acid amides.

In a further embodiment of the present invention, the targeted microbe is a bacteria, preferably a gram+bacteria.

The present invention also includes using carboxylic acid amides in a method of treating a disease or condition in a useful warm blooded mammal who has a microbial infection that is susceptible to treatment and who is in need of treatment with an antimicrobial effective amount of a carboxylic acid amide. The warm blooded mammal includes humans, farm animals such as horses, sheep, cattle, pigs and the alike as well as pets such as cats and dogs. It is preferred that the warm blooded mammal be a human.

In treating diseases or conditions in a warm blooded mammal, the carboxylic acid amides of the invention can be use alone or with other carboxylic acid amides of the invention as well as with other antibacterial agents or other pharmaceutical agents useful in treating the disease or condition.

To treat a systemic infection by a susceptible bacteria the carboxylic acid amides of the present invention are administered parenterally, orally, topically (transdermally) or rectally. Parental administration includes intravenous (IV), intramuscular, intradermal, intraperitoneal (IP) or subcutaneously (SQ). Parenteral administration requires a sterile isotonic aqueous solution buffered to an appropriate pH of the carboxylic acid amides or a suspension or emulsion for sustained release administration. Systemic infections can also be treated orally by solid dosage forms such as tablets, capsules, dispersible granules or lozenges and by liquid dosage forms such as solutions, syrups, suspensions and emulsions. These would include dosage forms for immediate release as well as sustained dosage forms for 12 or 24 hour administration. The carboxylic acid amides can be administered by way of a transdermal patch which is of particular benefit for those unable to swallow or where parenteral administration is not desirable. Further, the carboxylic acid amides can be formulated into suppositories for rectal administration which is of particular benefit for those unable to swallow or where parenteral administration is not desirable. It is known to those skilled in the art how to prepare the sterile parenteral formulations for parenteral administration, solid and liquid dosage forms for oral administrating, transdermal patches and suppositories of the carboxylic acid amides.

To treat a topical infection by a susceptible bacterial the carboxylic acid amides are administered in dosage forms suitable for topical administration including, but not limited to, creams, ointments, lotions, solutions, suspensions, emulsions and bandages impregnated with the carboxylic acid amides. It is known to those skilled in the art how to prepare the topical pharmaceutical dosage forms to administer the carboxylic acid amides.

Parenterally or orally, the carboxylic acid amides are given in an effective amount to stop the bacterial infection which is from about 1 to about 100 mg/kg/day, preferably from about 5 to about 50 mg/kg/day. Parenterally, the antibacterial carboxylic acid amides are given continuously by way of an IV or one to four times daily by injection. When infused IV it should be given at about 60 to 120 ml/hr depending on the concentration of the mixture being administered. Orally, the dose can be given once a day or divided into two or four doses a day. Topically, the topical formulation should have an effective amount of from about 0.05% to 5% of the carboxylic acid amides.

The exact dosage and frequency of administration depends on the particular carboxylic acid amide used, the particular condition being treated, the severity of the condition being treated, the age, weight, general physical condition of the particular patient, other medication the individual may and/or the patients response to the particular condition being treated as is known to those skilled in the art. With systemic infections physicians can monitor the progress of treatment by monitoring blood markers as well as the blood level of the antibacterial carboxylic acid amides as is known to those skilled in the art.

Having now generally described the invention, the same will be more readily understood through reference to the following Examples, which are provided by way of illustration, and are not intended to be limiting of the present invention, unless specified.

### Examples

### Example 1

### Treating a Systemic Infection in a Dog

A 15 kg dog with a badly infected wound is given 300 mg twice a day mixed in his food of trans-3-(naphth-2-yl)-but-2-enoic acid-N-(2-carboxyphenyl)amide. After 2 days the wound looks better and after 5 days it is almost healed.

### Example 2

### Treating a Topical Infection in a Human

A 50 kg 35 year old female is diagnosed by a dermatologist with a gram+bacterial infection on the skin of her left foot. He prescribed a 2.5% cream of 5-bromo-2-[[(2E)-3-(2-naphthalenyl)-1-oxo-2-butenyl]amino]benzoic acid which she applied twice daily. After about 4 days the infection was gone and the skin appears normal.

### Example 3

### Treating a Systemic Infection in a Human

A 70 kg 44 year old male has a 102° fever and a bad cough. An internist diagnoses his problem as a systemic gram+bacterial infection of S. aureus. He prescribes trans-3-(naphth-2-yl)but-2-enoic acid-N-(2-carboxyphenyl)amide with the directions to take two 300 mg tablets three daily which the patient does. After two days the fever comes down and after five days the temperature is normal and the cough is gone.

Having now fully described this invention, it will be appreciated by those skilled in the art that based upon the teachings herein, the same can be performed within a wide range of equivalent parameters, concentrations, and conditions without departing from the spirit and scope of the invention and without undue experimentation. Thus, changes and modifications may be made without departing from this subject matter described herein and its broader aspects and, therefore, the appended claims are to encompass within their scope all such changes and modifications as are within the true spirit and scope of this subject matter described herein.

What is claimed is:

1. A method of treating a disease or condition in a useful warm blooded mammal who has a microbial infection that is susceptible to treatment and who is in need of treatment with an antimicrobial effective amount of a carboxylic acid amide of the formula 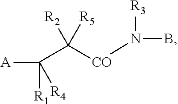
in which:
R₁ indicates a hydrogen atom, a C₁₋₃-alkyl or trifluoromethyl group, R₂ indicates a hydrogen, fluorine, chlorine or bromine atom, a C₁₋₃-alkyl, C₃₋₇-cycloalkyl or C₁₋₃-alkoxy group or also, if R₄ and R₅ each indicate a hydrogen atom, R₁ and R₂ together indicate an n-C₁₋₃-alkylene group which may be substituted by a C₁₋₃-alkyl group, R₃ indicates a hydrogen atom or a C₅-alkyl group, R₄ and R₅ each indicate a hydrogen atom or together indicate another carbon-carbon bond, A indicates a phenyl, naphthyl or tetrahydronaphthyl group substituted by a fluorine, chlorine, bromine or iodine atom, by a C₁₋₆-alkyl, C₃₋₇-cycloalkyl, phenyl, C₁₋₃-alkoxy, cyano, trifluoromethyl or nitro group, and the above described monosubstituted phenyl and naphthyl groups may also be substituted by a fluorine, chlorine or bromine atom, by a C₁₋₃-alkyl or C₁₋₃-alkoxy group and the above described disubstituted phenyl groups may also be substituted by a C₁₋₃-alkyl or C₁₋₃-alkoxy group, a naphthyl group, a chromane or chromene group in which a methylene group may be replaced by a carbonyl group, a 5- or 6-membered heteroaryl group which may be substituted in the carbon skeleton by a fluorine, chlorine or bromine atom, by a C₁₋₃-alkyl or C₁₋₃-alkoxy group, and the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms and the phenyl ring may also be substituted in the carbon skeleton by a fluorine, chlorine or bromine atom, by a C₁₋₃-alkyl or C₁₋₃-alkoxy group, a phenylvinyl group or R₁ together with A and the carbon atom between them indicates a C₅₋₇-cycloalkylidene group to which a phenyl ring may be fused by two adjacent carbon atoms, and the phenyl ring may also be substituted by one or two C₁₋₃-alkyl or C₁₋₃-alkoxy groups, and the substituents may be identical or different, and B indicates a 5- or 6-membered heteroaryl group substituted by a carboxy group or capable of being converted into a carboxy group in vivo, a phenyl or naphthyl group, each of which may be substituted by a carboxy group, by a group which may be converted into a carboxy group in vivo or by a group which is negatively charged under physiological conditions, and the above described phenyl groups may also be substituted by a fluorine, chlorine, bromine or iodine atom, by a C₁₋₃-alkyl, trifluoromethyl, phenyl, hydroxy, C₁₋₃-alkoxy, C₁₋₃-alkylsulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by a C₁₋₃-alkyl group which is substituted by a hydroxy, C₁₋₃-alkoxy, amino, C₁₋₄-alkylamino, di-(C₁₋₄-alkyl)-amino, C₃₋₇-cycloalkylamino, pyrrolidino, piperidino, morpholino, piperazino or N—(C₁₋₃-alkyl)-piperazino group, by an n-C₂₋₃-alkoxy, C₂₋₃-alkenyl or C₂₋₃-alkynyl group substituted in the 2 or 3 position by a di-(C₁₋₃-alkyl)-amino group, by an amino group, by an N—(C₁₋₃-alkyl)-amino or N,N-di-(C₁₋₃-alkyl)-amino group in which the alkyl moiety may in each case be substituted in the 2 or 3 position in relation to the nitrogen atom by a C₁₋₃-alkoxy group, by a N-phenylamino, N-(phenyl-C₁₋₃-alkyl)-amino or N-(pyridyl-C₁₋₃-alkyl)-amino group in which in each case a hydrogen atom of the above described amino groups may be substituted by a C₁₋₃-alkylsulphonyl, phenyl-C₁₋₃-alkylsulphonyl or phenylsulphonyl group or by a C₁₋₇-alkyl group, which may be replaced in the 2 to 5 position by a C₁₋₃-alkoxy, cyano, amino, C₁₋₃-alkylamino, di-(C₁₋₃-alkyl)-amino or tetrazolyl group, by an aminocarbonyl or C₁₋₃-alkylaminocarbonyl group which may in each case be substituted at the amino-nitrogen atom by a C₁₋₄-alkyl group which may be substituted by a vinyl, ethynyl, phenyl, pyridyl, imidazolyl, carboxy or trifluoromethyl group or, with the exception of the 2 position based on the aminocarbonyl nitrogen atom, by a hydroxy, C₁₋₃-alkoxy, C₁₋₃-alkylthio, amino, C₁₋₃-alkylamino, di-(C₁₋₃-alkyl)-amino, C₁₋₄-alkanoylamino or C₁₋₅-alkoxycarbonylamino group, by a C₃₋₇-cycloalkyl, C₅₋₉-Azabicycloalkyl, phenyl, pyridyl, C₁₋₃-alkoxy or di-(C₁₋₃-alkyl)-amino group, by a C₁₋₃-alkyl group which is substituted by a piperidin-3-yl or piperidin-4-yl group which may be substituted in the 1 position by a C₁₋₃-alkyl or C₁₋₅-alkoxycarbonyl group, or by an amino, C₁₋₃-alkylamino or phenyl-C₁₋₃-alkylamino group which may be substituted at the amino-nitrogen atom by a C₁₋₄-alkanoyl, C₁₋₅-alkoxycarbonyl, benzoyl, pyrrolidino, piperidino, morpholino or N—(C₁₋₃-alkyl)-piperazino group, by a carbonyl group substituted by a pyrrolidino, pyrrolino, piperidino, morpholino or N—(C₁₋₃-alkyl)-piperazino group, by a sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an amino or N—(C₁₋₃-alkyl)-amino group which is substituted in each case at the amino-nitrogen atom by an aminocarbonyl, C₁₋₃-alkylaminocarbonyl, phenyl-C₁₋₃-alkylaminocarbonyl, phenylaminocarbonyl, phenoxyphenylaminocarbonyl, pyridylaminocarbonyl, pyrrolidinocarbonyl, piperidinocarbonyl, morpholinocarbonyl or N—(C₁₋₃-alkyl)-piperazinocarbonyl group, and any hydrogen atom present in the above described aminocarbonyl groups may also be substituted by a C₁₋₃-alkyl group, by a 5- or 6-membered heteroaryl group, by a dihydro-oxazolyl, dihydro-imidazolyl, 2-oxo-pyrrolidino, 2-oxo-piperidino or 2-oxo-hexamethyleneimino group to which a phenyl ring may be fused by two adjacent carbon atoms, by an ethynyl group substituted by a phenyl, hydroxymethyl or dimethylamino group, and also the above described mono- or disubstituted phenyl groups may be substituted by another fluorine, chlorine or bromine atom or by one or two other C₁₋₃-alkyl or C₁₋₃-alkoxy groups and two C₁₋₃-alkoxy groups in the o position may be replaced by a methylenedioxy group, in particular R₁ indicates a hydrogen atom, a C₁₋₃-alkyl or trifluoromethyl group, R₂ indicates a hydrogen, fluorine, chlorine or bromine atom, a C₁₋₃-alkyl, C₃₋₇-cycloalkyl or C₁₋₃-alkoxy group or, if R₄ and R₅ each indicate a hydrogen atom, R₁ and R₂ together indicate an n-C₁₋₃-alkylene group which may be substituted by a C₁₋₃-alkyl group, R₃ indicates a hydrogen atom or a C₁₋₅-alkyl group, R₄ and R₅ each indicate a hydrogen atom or together indicate another carbon-carbon bond, A indicates a phenyl, naphthyl or tetrahydronaphthyl group substituted by a fluorine, chlorine, bromine or iodine atom, by a C₁₋₆-alkyl, C₃₋₇-cycloalkyl, phenyl, C₁₋₃-alkoxy, trifluoromethyl or nitro group, and the above described monosubstituted phenyl and naphthyl groups may also be substituted by a fluorine, chlorine or bromine atom, or by a C₁₋₃-alkyl or C₁₋₃-alkoxy group, a naphthyl group, a chromane or chromene group in which a methylene group may be replaced by a carbonyl group, a 5- or 6-membered heteroaryl group which may be substituted in the carbon skeleton by a fluorine, chlorine or bromine atom or by a C₁₋₃-alkyl or C₁₋₃-alkoxy group, and the 6-membered heteroaryl groups contain one, two or three nitrogen atoms and the 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms and the phenyl ring may also be substituted in the carbon skeleton by a fluorine, chlorine or bromine atom or by a C₁₋₃-alkyl or C₁₋₃-alkoxy group, a phenylvinyl group or R₁ together with A and the carbon atom between them indicate a C₅₋₇-cycloalkylidene group to which a phenyl ring may be fused by two adjacent carbon atoms, and the phenyl ring may also be substituted by one or two C₁₋₃-alkyl or C₁₋₃-alkoxy groups, and the substituents may be identical or different, and B indicates a phenyl, naphthyl or heteroaryl group, each of which may be substituted by a carboxy group, by a group which may be converted into a carboxy group in vivo or by a group which is negatively charged under physiological conditions, and the above described phenyl groups may also be substituted by a fluorine, chlorine, bromine or iodine atom, by a C₁₋₃-alkyl, hydroxy, C₁₋₃-alkoxy, C₁₋₃-alkylsulphonyloxy, phenylsulphonyloxy, carboxy, C₁₋₃-alkoxycarbonyl, formyl, C₁₋₃-alkylcarbonyl, C₁₋₃-alkylsulphonyl, phenylsulphonyl, nitro, pyrrolidino, piperidino, morpholino, N—(C₁₋₃-alkyl)-piperazino, aminosulphonyl, C₁₋₃-alkylaminosulphonyl or di-(C₁₋₃-alkyl)-aminosulphonyl group, by an n-C₂₋₃-alkoxy group substituted in the 2 or 3 position by a di-(C₁₋₃-alkyl)-amino group, by an amino, N—(C₁₋₃-alkyl)-amino, N-(phenyl-C₁₋₃-alkyl)-amino or N-(pyridyl-C₁₋₃-alkyl)-amino group in which in each case a hydrogen atom of the amino group may be substituted by a C₁₋₃-alkylsulphonyl or phenylsulphonyl group or by a C₁₋₇-alkyl group, which may be substituted in the 2 to 5 position by a C₁₋₃-alkoxy, cyano, amino, C₁₋₃-alkylamino, di-(C₁₋₃-alkyl)-amino or tetrazolyl group, by a carbonyl or sulphonyl group substituted by an amino, C₁₋₃-alkylamino, di-(C₁₋₃-alkyl)-amino, pyrrolidino, piperidino, morpholino or N—(C₁₋₃-alkyl)-piperazino group, by an imidazolyl or pyrazolyl group which may be substituted by a C₁₋₄-alkyl group, which may also be substituted by a C₁₋₃-alkyl, phenyl, trifluoromethyl or furyl group, and may also be substituted by another fluorine, chlorine or bromine atom, by another C₁₋₃-alkyl or C₁₋₃-alkoxy group, and the above described 6-membered heteroaryl groups contain one, two or three nitrogen atoms and the above described 5-membered heteroaryl groups contain an imino group which may be substituted by a C₁₋₃-alkyl group, an oxygen or sulphur atom or an imino group which may be substituted by a C₁₋₃-alkyl group and an oxygen or sulphur atom or one or two nitrogen atoms and also a phenyl ring may be fused to the above described monocyclic heteroaryl groups by two adjacent carbon atoms, and the phenyl ring may be substituted in the carbon skeleton by a fluorine, chlorine or bromine atom or by a C₁₋₃-alkyl or C₁₋₃-alkoxy group, and the above described 5-membered monocyclic heteroaryl groups in the carbon skeleton may also be substituted by a C₁₋₄-alkyl, trifluoromethyl, phenyl or furanyl group and by another C₁₋₃-alkyl group, and amino and imino groups mentioned in the definition of the above described groups may also be substituted by a group which can be cleaved in vivo, an isomer or a salt thereof.

2. A method of treating a disease or condition in a useful warm blooded mammal who has a microbial infection that is susceptible to treatment and who is in need of treatment with an antimicrobial effective amount of a carboxylic acid amide of the formula 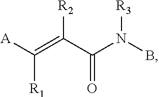
in which
R1 indicates a hydrogen atom, a C1-3-alkyl or trifluoromethyl group, R2 indicates a hydrogen, fluorine, chlorine or bromine atom or a C1-3-alkyl group, R3 indicates a hydrogen atom or a C1-5-alkyl group, A indicates a phenyl or naphthyl group substituted by a fluorine, chlorine, bromine or iodine atom, by a C1-6-alkyl, C3-7-cycloalkyl, phenyl, C1-3-alkoxy, cyano, trifluoromethyl or nitro group, and the above described monosubstituted phenyl and naphthyl groups may also be substituted by a fluorine, chlorine or bromine atom, by a C1-3-alkyl or C1-3-alkoxy group and the above described disubstituted phenyl groups may also be substituted by a C1-3-alkyl or C1-3-alkoxy group, a naphthyl group, a chromane or chromene group in which a methylene group may be replaced by a carbonyl group, or a 5- or 6-membered heteroaryl group which may be substituted in the carbon skeleton by a fluorine, chlorine or bromine atom or by a C1-3-alkyl or C1-3-alkoxy group, while the 6-membered heteroaryl groups contain one, two or three nitrogen atoms and the 5-membered heteroaryl groups contain an imino group which may be substituted by a C1-3-alkyl group, an oxygen or sulphur atom or an imino group which may be substituted by a C1-3-alkyl group and an oxygen or sulphur atom, or one or two nitrogen atoms and also a phenyl ring may be fused to the above described monocyclic heteroaryl groups by two adjacent carbon atoms and may also be substituted in the carbon skeleton by a fluorine, chlorine or bromine atom or by a C1-3-alkyl or C1-3-alkoxy group, and B indicates a phenyl or naphthyl group which is substituted in each case by a carboxy group, by a group which may be converted into a carboxy group in vivo or by a group which is negatively charged under physiological conditions and may which may be also be substituted by a fluorine, chlorine, bromine or iodine atom or by a C1-3-alkyl, trifluoromethyl or methoxy group, and the above described phenyl groups are also substituted by a C1-3-alkyl group which is substituted by an amino, C1-4-alkylamino, di-(C1-4-alkyl)-amino, C3-7-cycloalkylamino, pyrrolidino, piperidino, morpholino, piperazino or N—(C1-3-alkyl)-piperazino group, while the amino and imino groups mentioned in the definition of the above described groups may also be substituted by a group which can be cleaved in vivo, an isomer or a salt thereof.

3. A method of treating a disease or condition a useful warm blooded mammal who has a microbial infection that is susceptible to treatment and who is in need of treatment with an antimicrobial effective amount of a carboxylic acid amide of the formula 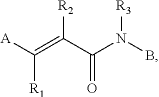
in which
R1 indicates a hydrogen atom or a C1-3-alkyl group, R2 indicates a hydrogen, fluorine, chlorine or bromine atom or a C1-3-alkyl group, R3 indicates a hydrogen atom or a C1-5-alkyl group, A indicates a chromane or chromene group linked by a fused-on phenyl ring in which a methylene group may be replaced by a carbonyl group, or a bicyclic heteroaryl group consisting of a 5- or 6-membered heteroaryl group which may be substituted in the carbon skeleton by a fluorine, chlorine or bromine atom or by a C1-3-alkyl or C1-3-alkoxy group, in which the 6-membered heteroaryl groups contain one, two or three nitrogen atoms and the 5-membered heteroaryl groups contain an imino group which may be substituted by a C1-3-alkyl group, an oxygen or sulphur atom, or an imino group which may be substituted by a C1-3-alkyl group and an oxygen or sulphur atom or one or two nitrogen atoms, and a phenyl ring fused to the above described monocyclic heteroaryl groups by two adjacent carbon atoms, by means of which the bicyclic heteroaryl group is linked to the R1-substituted alkene-carbon atom and which may also be substituted in the carbon skeleton by a fluorine, chlorine or bromine atom or by a C1-3-alkyl or C1-3-alkoxy group, and B indicates a 5- or 6-membered heteroaryl group substituted by a carboxy group or by a group which may be converted into a carboxy group in vivo or a phenyl or naphthyl group which is substituted in each case by a carboxy group, by a group which may be converted into a carboxy group in vivo or by a group which is negatively charged under physiological conditions, while the above described phenyl group may also be substituted by a fluorine, chlorine, bromine or iodine atom, by a C1-3-alkyl, trifluoromethyl, phenyl, hydroxy, C1-3-alkoxy, C1-3-alkyl-sulphonyloxy, phenylsulphonyloxy, carboxy, C1-3-alkoxycarbonyl, formyl, C1-3-alkylcarbonyl, C1-3-alkylsulphonyl, phenylsulphonyl, nitro, pyrrolidino, piperidino, morpholino, N—(C1-3-alkyl)-piperazino, aminosulphonyl, C1-3-alkylaminosulphonyl- or di-(C1-3-alkyl)-aminosulphonyl group, by an n-C2-3-alkoxy group substituted in the 2 or 3 position by a di-(C1-3-alkyl)-amino group, by an amino group, by an N—(C1-3-alkyl)-amino or N,N-di-(C1-3-alkyl)-amino group in which the alkyl moiety in the 2 or 3 position relative to the nitrogen atom may be substituted in each case by a C1-3-alkoxy group, by an N-phenylamino, N-(phenyl-C1-3-alkyl)-amino or N-(pyridyl-C1-3-alkyl)-amino group, by an aminocarbonyl group which may be mono- or disubstituted at the amino-nitrogen atom by a C1-3-alkyl group, by a pyrrolidinocarbonyl, piperidinocarbonyl, morpholinocarbonyl or N—(C1-3-alkyl)-piperazinocarbonyl group, by a sulphonyl group substituted by an amino, C1-3-alkylamino, di-(C1-3-alkyl)-amino, pyrrolidino, piperidino, morpholino or N—(C 1-3-alkyl)-piperazino group, by an amino or N—(C1-3-alkyl)-amino group which is substituted in each case at the amino-nitrogen atom by an aminocarbonyl, C1-3-alkylamino carbonyl, phenyl-C1-3-alkylaminocarbonyl, phenylaminocarbonyl, pyridylaminocarbonyl, pyrrolidinocarbonyl, piperidinocarbonyl, morpholinocarbonyl or N—(C1-3-alkyl)-piperazinocarbonyl group, and in the above described aminocarbonyl groups any hydrogen atom present may also be replaced by a C1-3-alkyl group, or by a 5 or 6-membered heteroaryl group, and the above described phenyl groups may also be substituted by another fluorine, chlorine or bromine atom or by another C1-3-alkyl or C1-3-alkoxy group and two C1-3-alkoxy groups in the o position may be replaced by a methylenedioxy group, and the above described 6-membered heteroaryl groups contain one, two or three nitrogen atoms and the above described 5-membered heteroaryl groups contain an imino group which may be substituted by a C1-3-alkyl group, an oxygen or sulphur atom, or an imino group which may be substituted by a C1-3-alkyl group and an oxygen or sulphur atom or one or two nitrogen atoms, an isomer or a salt thereof.

4. A method of treating a disease or condition in a useful warm blooded mammal according to claims 1-3 in which the microbe is a gram-positive bacteria.

5. A method of treating a disease or condition in a useful warm blooded mammal according to claims 1-3 in which the microbe is selected from the group consisting of: Staphylococcus aureus, Bacillus subtilis, Staphylococcus epidermidis, Streptococcus pneumoniae, Micrococcus leuteus, and Mycobacterium smegmatis.

6. A method of treating a disease or condition in a useful warm blooded mammal according to claim 5 in which the microbe is Staphylococcus aureus.

7. A method of treating a disease or condition in a useful warm blooded mammal according to claim 6 where the Staphylococcus aureus is methicillin resistant S. aureus (MRSA).

8. A method of treating a disease or condition in a useful warm blooded mammal according to claims 1-3 in which the useful warm blooded mammal is selected from the group consisting of humans, horses, sheep, cattle, pigs, cats and dogs.

9. A method of treating a disease or condition in a useful warm blooded mammal according to claim 8 in which useful warm blooded mammal is a human.

10. A method of treating a disease or condition in a useful warm blooded mammal according to claims 1-3 where the carboxylic acid amide is selected from the group consisting of
trans-3-(benzothien-6-yl)-but-2-enoic acid-N-(2-carboxy-phenyl)-amide,
trans-3-(benzothien-5-yl)-but-2-enoic acid-N-(2-carboxy-phenyl)-amide,
trans-3-(benzothien-6-yl)-but-2-enoic acid-N-(2-carboxy-4,5-dimethoxyphenyl)-amide,
trans-3-(benzothien-6-yl)-but-2-enoic acid-N-(2-carboxy-6-methylphenyl)-amide,
trans-3-(benzothien-6-yl)-but-2-enoic acid-N-(2-carboxy-4-fluorophenyl)-amide,
trans-3-(quinolin-6-yl)-but-2-enoic acid-N-(2-carboxy-phenyl)-amide,
trans-3-(naphtha-2-yl)but-2-enoic acid-N-(2-carboxyphenyl)-amide,
trans-4-bromocinnamic acid-N-(2-carboxyphenyl)-amide,
trans-2-methylcinnamic acid-N-(2-carboxyphenyl)-amide,
trans-4-methylcinnamic acid-N-(2-carboxyphenyl)-amide,
trans-4-trifluoromethylcinnamic acid-N-(2-carboxyphenyl)-amide,
trans-4-chlorocinnamic acid-N-(2-carboxyphenyl)-amide,
trans-2-nitrocinnamic acid-N-(2-carboxyphenyl)-amide,
trans-4-nitrocinnamic acid-N-(2-carboxyphenyl)-amide,
trans-3-(furan-2-yl)prop-2-enoic acid-N-(2-carboxyphenyl)-amide,
trans-3-(3′,4′dichlorophenyl)but-2-enoic acid-N-(2-carboxyphenyl)-amide,
trans-3,4-dichlorocinnamic acid-N-(2-carboxyphenyl)-amide,
trans-3-(biphenyl-4-yl)but-2-enoic acid-N-(2-carboxyphenyl)-amide,
trans-3-(naphtha-2-yl)prop-2-enoic acid-N-(2-carboxyphenyl)-amide,
trans-cinnamic acid-N-(2-carboxyphenyl)-amide,
trans-3-(phenyl)pent-2-enoic acid-N-(2-carboxyphenyl)-amide,
trans-4-methoxycinnamic acid-N-(2-carboxyphenyl)-amide,
trans-3-(naphtha-2-yl)prop-2-enoic acid-N-methyl-N-(2-carboxyphenyl)-amide,
trans-3-methoxy-4-benzoxycinnamic acid-N-(2-carboxyphenyl)-amide and
trans-5-bromo-2-[[(2E)-3-(2-naphthalenyl)-1-oxo-2-butenyl]amino]benzoic acid.

11. A method of treating a disease or condition in a useful warm blooded mammal according to claim 10 in which the carboxylic acid amide is trans-3-(naphth-2-yl)but-2-enoic acid-N-(2-carboxyphenyl)amide.
